(19) United States
(12) Patent Application Publication
(10) Pub. No.: US 2014/0141090 A1
(43) Pub. Date: May 22, 2014

(54) Pharmaceutical Composition Comprising Opioid Agonist And Sequestered Antagonist

(71) Applicant: Edward S. Wilson, Cary, NC (US)

(72) Inventor: Edward S. Wilson, Cary, NC (US)

(21) Appl. No.: 13/982,890

(22) PCT Filed: Jan. 25, 2012
(86) PCT No.: PCT/IB2012/050348

§ 371 Date: Jan. 23, 2014

(30) Foreign Application Priority Data
Feb. 2, 2011 (US) 61438882

(51) Int. Cl. A61K 9/50; A61K 31/485

(52) U.S. Cl. CPC A61K 9/5078; A61K 31/485
USPC 424/495; 514/282

(57) ABSTRACT

This invention pertains to pharmaceutical composition comprising a plurality of multi-layered beads having an oxycodone layer and a sequestering subunit comprising a naltrexone and a blocking agent, in particular pharmaceutical compositions comprising a higher level of naltrexone, and related compositions and methods of use, such as in the prevention of abuse of a therapeutic agent. The compositions of the present invention also have a long Tₘₐₓ for oxycodone release and a flatter release profile of oxycodone over time.

### FIELD OF THE DISCLOSURE

This disclosure pertains to pharmaceutical composition comprising a plurality of multi-layered beads having an oxycodone layer and a sequestering subunit comprising a naltrexone and a blocking agent, in particular pharmaceutical compositions comprising a higher level of naltrexone, and related compositions and methods of use, such as in the prevention of abuse of a therapeutic agent. The compositions described herein also have a long Tₘₐₓ for oxycodone release and a flatter release profile of oxycodone over time.

### BACKGROUND INFORMATION

Opioids, also called opioid agonists, are a class of drugs that exhibit opium-like or morphine-like properties. The opioids are employed primarily as moderate to strong analgesics, but have many other pharmacological effects as well, including drowsiness, respiratory depression, changes in mood, and mental clouding without a resulting loss of consciousness. Because of these other pharmacological effects, opioids have become the subject of dependence and abuse. Therefore, a major concern associated with the use of opioids is the diversion of these drugs from the illicit user, e.g., an addict.

Previous attempts to control the abuse potential associated with opioid analgesics include, for example, the combination of pentazocine and naloxone in tablets, commercially available in the United States as Talwin®Nx from Sanofi-Winthrop, Canterbury, Australia. Talwin®Nx contains pentazocine hydrochloride equivalent to 50 mg base and naloxone hydrochloride equivalent to 0.5 mg base. Talwin®Nx is indicated for the relief of moderate to severe pain. The amount of naloxone present in this combination has low activity when taken orally, and minimally interferes with the pharmacologic action of pentazocine. However, this amount of naloxone given parenterally has profound antagonistic action to narcotic analgesics. Thus, the inclusion of naloxone is intended to curb a form of misuse of oral pentazocine, which occurs when the dosage form is solubilized and injected. Therefore, this dosage has lower potential for parenteral misuse than previous oral pentazocine formulations. However, it is still subject to patient misuse and abuse by the oral route, for example, by the patient taking multiple doses at once. A fixed combination therapy comprising tilidine (50 mg) and naloxone (4 mg) has been available in Germany for the management of severe pain since 1978 (Valoron®N, Goedecke). The rationale for the combination of these drugs is effective pain relief and the prevention of tilidine addiction through naloxone-induced antagonisms at the tilidine receptors. A fixed combination of buprenorphine and naloxone was introduced in 1991 in New Zealand (Terngesic®Nx, Reckitt & Colman) for the treatment of pain.

International Patent Application No. PCT/US01/04346 (WO 01/58451) to Euroceltique, S.A., describes the use of a pharmaceutical composition that contains a substantially non-releasing opioid antagonist and a releasing opioid agonist as separate subunits that are combined into a pharmaceutical dosage form, e.g., tablet or capsule. However, because the agonist and antagonist are in separate subunits, they can be readily separated. Further, providing the agonist and antagonist as separate subunits, tablets are more difficult to form due to the mechanical sensitivity of some subunits comprising a sequestering agent.

The benefits of the abuse-resistant dosage form are especially great in connection with oral dosage forms of strong opioid agonists (e.g., morphine, hydromorphone, oxycodone or hydrocodone), which provide valuable analgesics but are prone to being abused. This is particularly true for sustained-release opioid agonist products, which have a large dose of a desirable opioid agonist intended to be released over a period of time in each dosage unit. Drug abusers take such sustained release product and crush, grind, extract or otherwise damage the product so that the full contents of the dosage form become available for immediate absorption.

Such abuse-resistant, sustained-release dosage forms have been described in the art (see, for example, U.S. Application Nos. 2003/0124185 and 2003/0044458). However, it is believed that substantial amounts of the opioid antagonist or other antagonist found in these sequestered forms are released over time (usually less than 24 hours) due to the osmotic pressure that builds up in the core of the sequestered form, as water permeates through the sequestered form into the core. The high osmotic pressure inside the core of the sequestered form causes the opioid antagonist or antagonist to be pushed out of the sequestered form, thereby causing the opioid antagonist or antagonist to be released from the sequestered form. To the extent that opioid antagonists have been sequestered for any extended length of time, the amount of antagonist sequestered relative to the sequestering subunit has been small. For example, U.S. Pat. No. 6,696,088 describes a sequestering subunit containing 2.3% naltrexone (3.3 mg out of a total of 140 mg). Furthermore, this formulation released 33% of the naltrexone within 36 hours when subjected to the USP Type II Paddle test and in vitro method of dissolution. US Patent Application No. 2010/0098771 describes a sequestering subunit containing 2.1% naltrexone with 5.7% leakage after 24 hours. U.S. Pat. No. 7,682,633 provides sequestration of the antagonist, but the antagonist is 2.6% of the sequestering subunit.

Furthermore, the amount of opioid antagonist sequestered in the prior art forms of abuse-resistant, sustained release dosage forms has been limited by the leakage of opioid antagonist from the dosage form when large quantities of opioid antagonist is sequestered. See for example, US Patent Application No. 2003/0004177.

In view of the foregoing drawbacks of the sequestered forms of the prior art, there exists a need in the art for a sequestered form of an opioid antagonist which provides for large amounts of antagonist to be sequestered wherein the antagonist is not substantially released from the sequestered form for long periods of time. Such a sequestered form of an opioid antagonist is disclosed herein. This and other objects and advantages of the disclosed subject matter, as well as additional features, will be apparent from the description provided herein.

### BRIEF SUMMARY OF THE DISCLOSURE

Provided herein are pharmaceutical compositions comprising an antagonist, an agonist, a seal coat, and a sequestering polymer, wherein the antagonist, agonist, seal coat and at least one sequestering polymer are all components of a single unit, and wherein the seal coat forms a layer physically separating the antagonist from the agonist from one another. Methods for manufacturing such a pharmaceutical composition are also provided. The pharmaceutical compositions described herein provide for sequestration of larger amounts of opioid antagonists than that of the prior art.

This disclosure provides compositions which comprises a plurality of multi-layer pellets comprising a water soluble core; an antagonist containing layer comprising naltrexone HCl coating the core; a sequestering polymer layer coating the antagonist containing layer; an agonist layer comprising an opioid agonist coating the sequestering polymer layer; and a controlled release layer coating the agonist layer; wherein the naltrexone HCl comprises at least 10% wt to wt of the opioid agonist and wherein the agonist is substantially released and the naltrexone HCl is substantially sequestered upon administration to a human being.

Also provided herein are compositions comprising a plurality of multi-layer pellets comprising a water soluble core; an antagonist containing layer comprising naltrexone HCl coating the core; a sequestering polymer layer coating the antagonist containing layer; an agonist layer comprising an opioid agonist coating the sequestering polymer layer; and a controlled release layer coating the agonist layer; wherein the weight of the naltrexone HCl comprises at least 5% the combined weight of the water soluble core, antagonist layer and sequestering polymer layer and wherein the agonist is substantially released and the naltrexone HCl is substantially sequestered upon administration to a human being.

### BRIEF DESCRIPTION OF THE DRAWINGS

FIG. 1. Graphical representation of mean oxycodone plasma concentration-time profiles for immediate release oxycodone and extended release oxycodone/naltrexone composition.

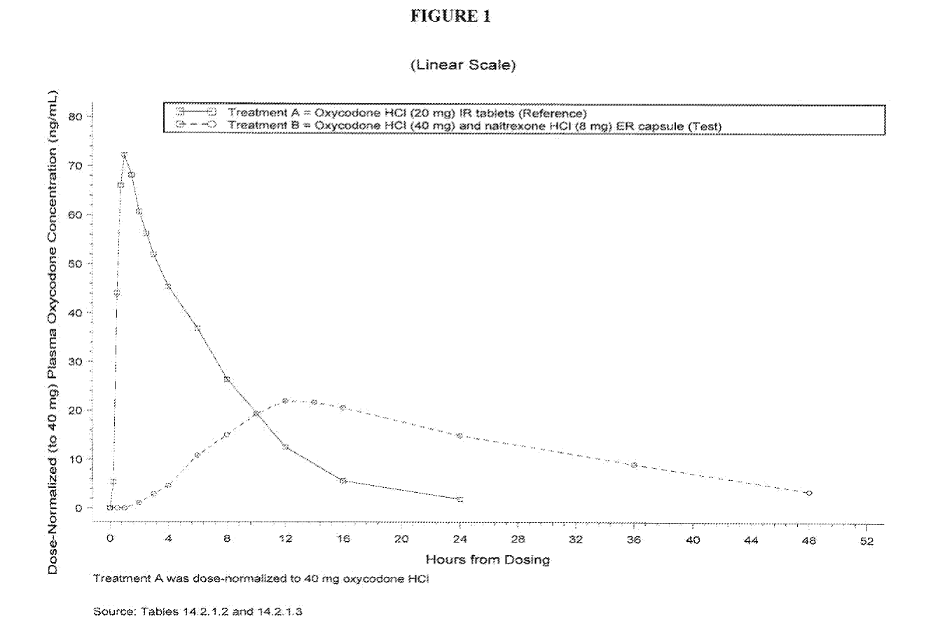

FIG. 2. Graphical representation of mean does normalized oxycodone plasma concentration-time profiles for immediate release oxycodone and extended release oxycodone/naltrexone composition.

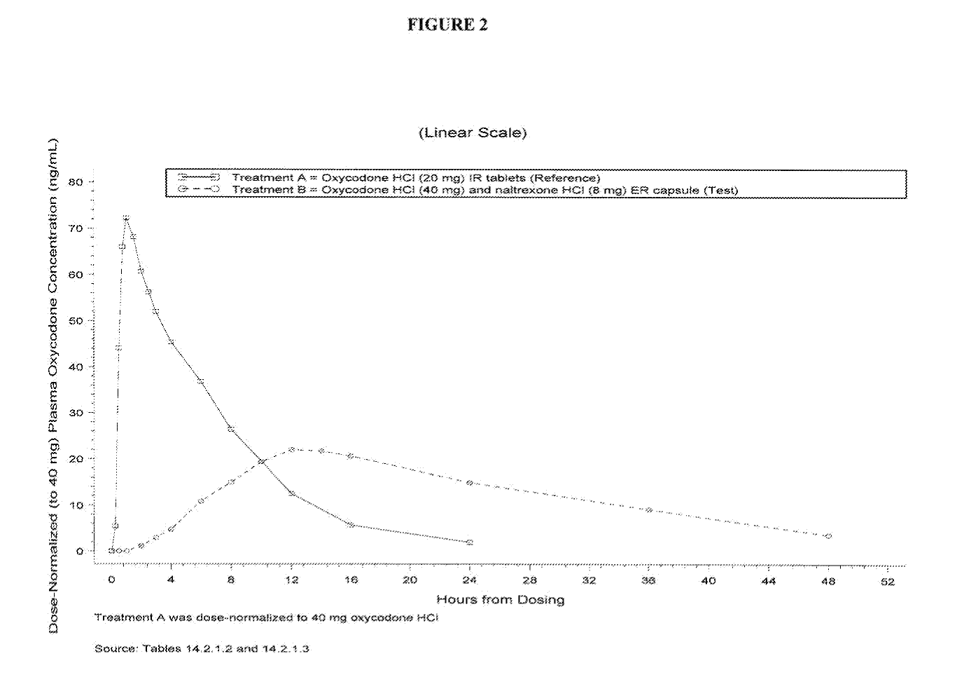

FIG. 3. Graphical representation of mean noroxycodone plasma concentration-time profiles for immediate release oxycodone and extended release oxycodone/naltrexone composition.

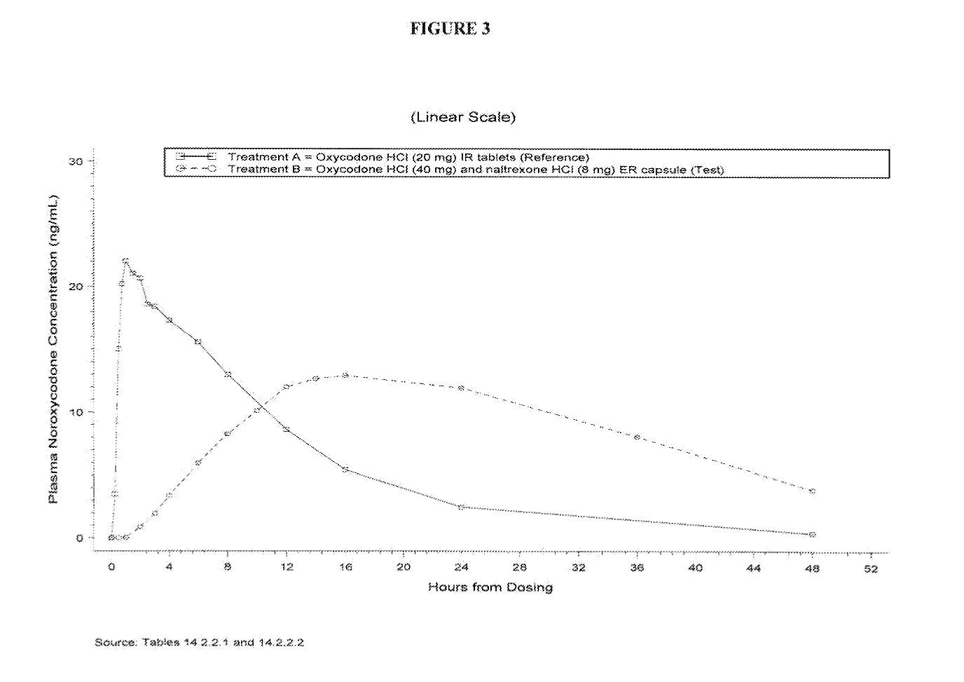

FIG. 4. Graphical representation of Drug Liking Bipolar VAS Mean of Raw Scores (Evaluable Population).

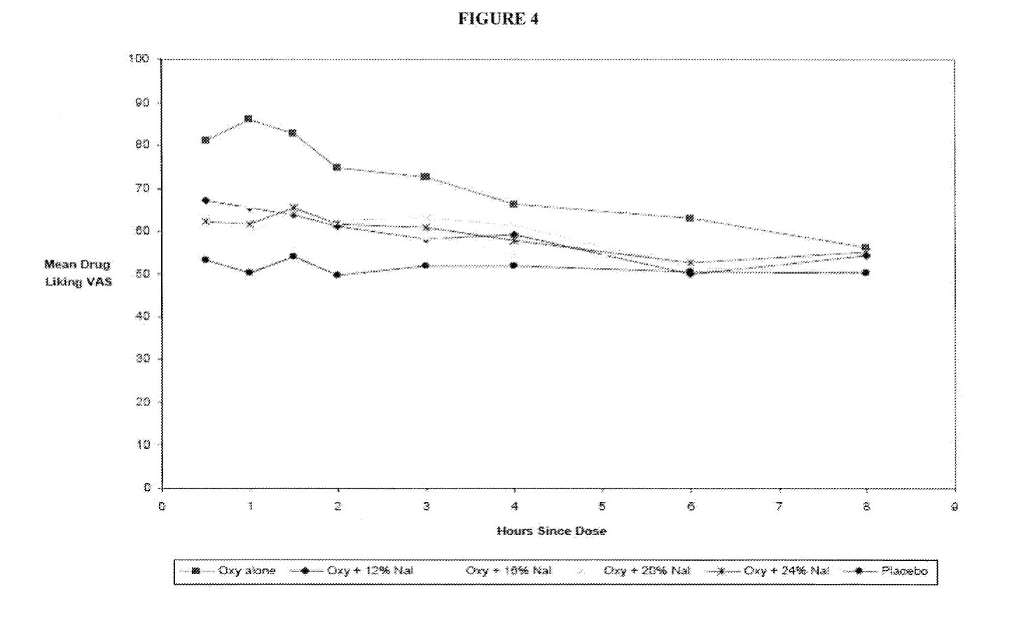

### DETAILED DESCRIPTION OF THE DISCLOSURE

Provided herein are compositions and methods for administering a multiple active agents to a mammal in a form and manner that minimizes the effects of either active agent upon the other in vivo. In certain embodiments, at least two active agents are formulated as part of a pharmaceutical composition. A first active agent may provide a therapeutic effect in vivo. The second active agent may be an antagonist of the first active agent, and may be useful in preventing misuse of the composition. For instance, where the first active agent is an opioid, the second active agent may be an antagonist of the opioid. The composition remains intact during normal usage by patients and the antagonist is not released. However, upon tampering with the composition, the antagonist may be released thereby preventing the opioid from having its intended effect. In certain embodiments, the active agents are both contained within a single unit, such as a bead, in the form of layers. The active agents may be formulated with a substantially impermeable barrier as, for example, a controlled-release composition, such that release of the antagonist from the composition is minimized. In certain embodiments, the antagonist is released in in vitro assays but is substantially not released in vivo. In vitro and in vive release of the active agent from the composition may be measured by any of several well-known techniques. For instance, in vivo release may be determined by measuring the plasma levels of the active agent or metabolites thereof (i.e., AUC, Cₘₐₓ).

In certain embodiments, one of the active agents is an opioid receptor agonist. Several opioid agonists are commercially available or in clinical trials and may be administered as described herein such that the alcohol effects are minimized. Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. Equianalgesic doses of these opioids, in comparison to a 15 mg dose of hydrocodone, are as follows: oxycodone (13.5 mg), codeine (90.0 mg), hydrocodone (15.0 mg), hydromorphone (3.375 mg), levorphanol (1.8 mg), meperidine (135.0 mg), methadone (9.0 mg), and morphine (27.0 mg).

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug. Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride. This disclosure is contemplated to encompass all such formulations, with the inclusion of an opioid antagonist and/or antagonist in sequestered form as part of a subunit comprising an opioid agonist.

Oral hydromorphone is commercially available in the United States, e.g., as Dilaudid® from Abbott Laboratories (Chicago, Ill.). Oral morphine is commercially available in the United States, e.g., as Kadian® from Faulding Laboratories (Piscataway, N.J.).

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms described herein may include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 5 mg to about 200 mg oxycodone. Preferred embodiments of the dosage forms may include 10 mg, 20 mg, 40 mg, 60 mg, 80 mg, 100 mg and 120 mg of oxycodone or a pharmaceutically acceptable salt thereof. Controlled release oxycodone formulations are known in the art. The following documents describe various controlled-release oxycodone formulations suitable for use as described herein, and processes for their manufacture: for example, U.S. Pat. Nos. 5,266,331; 5,549,912; 5,508,042; and 5,656,295, which are incorporated herein by reference. The opioid agonist can comprise tramadol and the sustained-release oral dosage forms can include from about 25 mg to 800 mg tramadol per dosage unit.

In certain embodiments, another active agent contained within the composition may be an opioid receptor antagonist. In certain embodiments, the agonist and antagonist are administered together, either separately or as part of a single pharmaceutical unit. In the instance when the therapeutic agent is an opioid agonist, the antagonist preferably is an opioid antagonist, such as naltrexone, naloxone, nalmefene, cyclazacine, levallorphan, derivatives or complexes thereof, pharmaceutically acceptable salts thereof, and combinations thereof. More preferably, the opioid antagonist is naloxone or naltrexone. By “opioid antagonist” is meant to include one or more opioid antagonists, either alone or in combination, and is further meant to include partial antagonists, pharmaceutically acceptable salts thereof, stereoisomers thereof, ethers thereof, esters thereof, and combinations thereof. In a preferred embodiment, when the antagonist is naltrexone, it is preferable that the intact dosage form releases less than 0.125 mg or less within 24 hours, with 0.25 mg or greater of naltrexone released after 1 hour when the dosage form is crushed or chewed.

In a preferred embodiment, the opioid antagonist comprises naltrexone. In the treatment of patients previously addicted to opioids, naltrexone has been used in large oral doses (over 100 mg) to prevent euphorigenic effects of opioid agonists. Naltrexone has been reported to exert strong preferential blocking action against mu over delta sites. Naltrexone is known as a synthetic congener of oxymorphone with no opioid agonist properties, and differs in structure from oxymorphone by the replacement of the methyl group located on the nitrogen atom of oxymorphone with a cyclopropylmethyl group. The hydrochloride salt of naltrexone is soluble in water up to about 100 mg/cc. The pharmacological and pharmacokinetic properties of naltrexone have been evaluated in multiple animal and clinical studies. See, e.g., Gonzalez et al. Drugs 35:192-213 (1988). Following oral administration, naltrexone is rapidly absorbed (within 1 hour) and has an oral bioavailability ranging from 5-40%. Naltrexone's protein binding is approximately 21% and the volume of distribution following single-dose administration is 16.1 L/kg.

Naltrexone is commercially available in tablet form (Revia®, DuPont (Wilmington, Del.)) for the treatment of alcohol dependence and for the blockade of exogenously administered opioids. See, e.g., Revia (naltrexone hydrochloride tablets), Physician's Desk Reference, 51^{st} ed., Montvale, N.J.; and Medical Economics 51:957-959 (1997). A dosage of 50 mg Revia® blocks the pharmacological effects of 25 mg IV administered heroin for up to 24 hours. It is known that, when coadministered with morphine, heroin or other opioids on a chronic basis, naltrexone blocks the development of physical dependence to opioids. It is believed that the method by which naltrexone blocks the effects of heroin is by competitively binding at the opioid receptors. Naltrexone has been used to treat narcotic addiction by complete blockade of the effects of opioids. It has been found that the most successful use of naltrexone for a narcotic addiction is with narcotic addicts having good prognosis, as part of a comprehensive occupational or rehabilitative program involving behavioral control or other compliance-enhancing methods. For treatment of narcotic dependence with naltrexone, it is desirable that the patient be opioid-free for at least 7-10 days. The initial dosage of naltrexone for such purposes has typically been about 25 mg, and if no withdrawal signs occur, the dosage may be increased to 50 mg per day. A daily dosage of 50 mg is considered to produce adequate clinical blockade of the actions of parenterally administered opioids. Naltrexone also has been used for the treatment of alcoholism as an adjunct with social and psychotherapeutic methods.

Other preferred opioid antagonists include, for example, cyclazocine and naltrexone, both of which have cyclopropylmethyl substitutions on the nitrogen, retain much of their efficacy by the oral route, and last longer, with durations approaching 24 hours after oral administration.

In one embodiment, a sequestering subunit comprising an opioid antagonist and a blocking agent, wherein the blocking agent substantially prevents release of the opioid antagonist from the sequestering subunit in the gastrointestinal tract for a time period that is greater than 24 hours is provided. This sequestering subunit is incorporated into a single pharmaceutical unit that also includes an opioid agonist. The pharmaceutical unit thus includes a core portion to which the opioid antagonist is applied. A seal coat is then optionally applied upon the antagonist. Upon the seal coat is then applied a composition comprising the pharmaceutically active agent. An additional layer containing the same or a different blocking agent may then be applied such that the opioid agonist is released in the digestive tract over time (i.e., controlled release). Thus, the opioid antagonist and the opioid agonist are both contained within a single pharmaceutical unit, which is typically in the form of a bead.

The term “sequestering subunit” as used herein refers to any means for containing an antagonist and preventing or substantially preventing the release thereof in the gastrointestinal tract when intact, i.e., when not tampered with. The term “blocking agent” as used herein refers to the means by which the sequestering subunit is able to prevent substantially the antagonist from being released. The blocking agent may be a sequestering polymer, for instance, as described in greater detail below.

The terms “substantially prevents,” “prevents,” or any words stemming therefrom, as used herein, means that the antagonist is substantially not released from the sequestering subunit in the gastrointestinal tract. By “substantially not released” is meant that the antagonist may be released in a small amount, but the amount released does not affect or does not significantly affect the analgesic efficacy when the dosage form is orally administered to a host, e.g., a mammal (e.g., a human), as intended. The terms “substantially prevents,” “prevents,” or any words stemming therefrom, as used herein, does not necessarily imply a complete or 100% prevention. Rather, there are varying degrees of prevention of which one of ordinary skill in the art recognizes as having a potential benefit. In this regard, the blocking agent substantially prevents or prevents the release of the antagonist to the extent that at least about 80% of the antagonist is prevented from being released from the sequestering subunit in the gastrointestinal tract for a time period that is greater than 24 hours. Preferably, the blocking agent prevents release of at least about 90% of the antagonist from the sequestering subunit in the gastrointestinal tract for a time period that is greater than 24 hours. More preferably, the blocking agent prevents release of at least about 95% of the antagonist from the sequestering subunit. Most preferably, the blocking agent prevents release of at least about 99% of the antagonist from the sequestering subunit in the gastrointestinal tract for a time period that is greater than 24 hours.

For purposes of this disclosure, the amount of the antagonist released after oral administration can be measured in-vitro by dissolution testing as described in the United States Pharmacopeia (USP26) in chapter <711> Dissolution. For example, using 900 mL of 0.1 N HCl, Apparatus 2 (Paddle), 75 rpm, at 37° C. to measure release at various times from the dosage unit. Other methods of measuring the release of an antagonist from a sequestering subunit over a given period of time are known in the art (see, e.g., USP26).

Without being bound to any particular theory, it is believed that the sequestering subunit described herein overcomes the limitations of the sequestered forms of an antagonist known in the art in that the sequestering subunit described herein reduces osmotically-driven release of the antagonist from the sequestering subunit. Furthermore, it is believed that the present inventive sequestering subunit reduces the release of the antagonist for a longer period of time (e.g., greater than 24 hours) in comparison to the sequestered forms of antagonists known in the art. The fact that the sequestered subunit described herein may provide a longer prevention of release of the antagonist is particularly relevant, since precipitated withdrawal could occur after the time for which the therapeutic agent is released and acts. It is well known that the gastrointestinal tract transit time for individuals varies greatly within the population. Hence, the residue of the dosage form may be retained in the tract for longer than 24 hours, and in some cases for longer than 48 hours. It is further well known that opioid analgesics cause decreased bowel motility, further prolonging gastrointestinal tract transit time. Currently, sustained-release forms having an effect over a 24 hour time period have been approved by the Food and Drug Administration. In this regard, the present inventive sequestering subunit provides prevention of release of the antagonist for a time period that is greater than 24 hours when the sequestering subunit has not been tampered.

The sequestering subunit described herein is designed to prevent substantially the release of the antagonist when intact. By “intact” is meant that a dosage form has not undergone tampering. The term “tampering” is meant to include any manipulation by mechanical, thermal and/or chemical means, which changes the physical properties of the dosage form. The tampering can be, for example, crushing, shearing, grinding, chewing, dissolution in a solvent, heating (for example, greater than about 45° C.), or any combination thereof. When the sequestering subunit described herein has been tampered with, the antagonist may be immediately released from the sequestering subunit.

By “subunit” is meant to include a composition, mixture, particle; etc., that can provide a dosage form (e.g., an oral dosage form) when combined with another subunit. The subunit can be in the form of a bead, pellet, granule, spheroid, or the like, and can be combined with additional same or different subunits, in the form of a capsule, tablet or the like, to provide a dosage form, e.g., an oral dosage form. The subunit may also be part of a larger, single unit, forming part of that unit, such as a layer. For instance, the subunit may be a core coated with an antagonist and a seal coat; this subunit may then be coated with additional compositions including a pharmaceutically active agent such as an opioid agonist.

The blocking agent prevents or substantially prevents the release of the antagonist in the gastrointestinal tract for a time period that is greater than 24 hours, e.g., between 24 and 25 hours, 30 hours, 35 hours, 40 hours, 45 hours, 48 hours, 50 hours, 55 hours, 60 hours, 65 hours, 70 hours, 72 hours, 75 hours, 80 hours, 85 hours, 90 hours, 95 hours, or 100 hours; etc. Preferably, the time period for which the release of the antagonist is prevented or substantially prevented in the gastrointestinal tract is at least about 48 hours. More preferably, the blocking agent prevents or substantially prevents the release for a time period of at least about 72 hours.

The blocking agent of the present inventive sequestering subunit can be a system comprising a first antagonist-impermeable material and a core. By “antagonist-impermeable material” is meant any material that is substantially impermeable to the antagonist, such that the antagonist is substantially not released from the sequestering subunit. The term “substantially impermeable” as used herein does not necessarily imply complete or 100% impermeability. Rather, there are varying degrees of impermeability of which one of ordinary skill in the art recognizes as having a potential benefit. In this regard, the antagonist-impermeable material substantially prevents or prevents the release of the antagonist to an extent that at least about 80% of the antagonist is prevented from being released from the sequestering subunit in the gastrointestinal tract for a time period that is greater than 24 hours. Preferably, the antagonist-impermeable material prevents release of at least about 90% of the antagonist from the sequestering subunit in the gastrointestinal tract for a time period that is greater than 24 hours. More preferably, the antagonist-impermeable material prevents release of at least about 95% of the antagonist from the sequestering subunit. Most preferably, the antagonist-impermeable material prevents release of at least about 99% of the antagonist from the sequestering subunit in the gastrointestinal tract for a time period that is greater than 24 hours. The antagonist-impermeable material prevents or substantially prevents the release of the antagonist in the gastrointestinal tract for a time period that is greater than 24 hours, and desirably, at least about 48 hours. More desirably, the antagonist-impermeable material prevents or substantially prevents the release of the adversive agent from the sequestering subunit for a time period of at least about 72 hours.

Preferably, the first antagonist-impermeable material comprises a hydrophobic material, such that the antagonist is not released or substantially not released during its transit through the gastrointestinal tract when administered orally as intended, without having been tampered with. Suitable hydrophobic materials for use as described herein may include those set forth below. The hydrophobic material is preferably a pharmaceutically acceptable hydrophobic material. Preferably, the pharmaceutically acceptable hydrophobic material comprises a cellulose polymer.

It is preferred that the first antagonist-impermeable material comprises a polymer insoluble in the gastrointestinal tract. One of ordinary skill in the art appreciates that a polymer that is insoluble in the gastrointestinal tract will prevent the release of the antagonist upon ingestion of the sequestering subunit. The polymer can be a cellulose or an acrylic polymer. Desirably, the cellulose is selected from the group consisting of ethylcellulose, cellulose acetate, cellulose propionate, cellulose acetate propionate, cellulose acetate butyrate, cellulose acetate phthalate, cellulose triacetate, and combinations thereof. Ethylcellulose includes, for example, one that has an ethoxy content of about 44 to about 55%. Ethylcellulose can be used in the form of an aqueous dispersion, an alcoholic solution, or a solution in other suitable solvents. The cellulose can have a degree of substitution (D.S.) on the anhydroglucose unit, from greater than zero and up to 3 inclusive. By “degree of substitution” is meant the average number of hydroxyl groups on the anhydroglucose unit of the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, monocellulose alkanylate, dicellulose alkanylate, tricellulose alkanylate, monocellulose alkenylates, dicellulose alkenylates, tricellulose alkenylates, monocellulose aroylates, dicellulose aroylates, and tricellulose aroylates.

More specific celluloses include cellulose propionate having a D.S. of 1.8 and a propyl content of 39.2 to 45 and a hydroxy content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxy content of 0.5 to 4.7%; cellulose triacylate having a D.S. of 2.9 to 3, such as cellulose triacetate, cellulose trivalerate, cellulose trilaurate, cellulose tripatmitate, cellulose trisuccinate, and cellulose trioctanoate; cellulose diacylates having a D.S. of 2.2 to 2.6, such as cellulose disuccinate, cellulose dipalmitate, cellulose dioctanoate, cellulose dipentanoate, and coesters of cellulose, such as cellulose acetate butyrate, cellulose acetate octanoate butyrate, and cellulose acetate propionate.

Additional cellulose polymers that may be useful for preparing a sequestering subunit described herein may include acetaldehyde dimethyl cellulose acetate, cellulose acetate ethylcarbamate, cellulose acetate methycarbamate, and cellulose acetate dimethylaminocellulose acetate.

The acrylic polymer preferably is selected from the group consisting of methacrylic polymers, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and combinations thereof. An acrylic polymer useful for preparation of a sequestering subunit described herein may include acrylic resins comprising copolymers synthesized from acrylic and methacrylic acid esters (e.g., the copolymer of acrylic acid lower alkyl ester and methacrylic acid lower alkyl ester) containing about 0.02 to about 0.03 mole of a tri(lower alkyl) ammonium group per mole of the acrylic and methacrylic monomer used. An example of a suitable acrylic resin is ammonio methacrylate copolymer NF21, a polymer manufactured by Rohm Pharma GmbH, Darmstadt, Germany, and sold under the Eudragit® trademark. Eudragit RS30D is preferred. Eudragit® is a water-insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins, such as Eudragit®, can be used in the form of an aqueous dispersion or as a solution in suitable solvents.

In another preferred embodiment, the antagonist-impermeable material is selected from the group consisting of polylactic acid, polyglycolic acid, a co-polymer of polylactic acid and polyglycolic acid, and combinations thereof. In certain other embodiments, the hydrophobic material includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesters, polydioxanone, polygluconate, poly lactic-acid-polyethylene oxide copolymers, poly(hydroxybutyrate), polyphosphoester or combinations thereof.

Preferably, the biodegradable polymer comprises a poly(lactic/glycolic acid), a copolymer of lactic and glycolic acid, having a molecular weight of about 2,000 to about 500,000 daltons. The ratio of lactic acid to glycolic acid is preferably from about 100:1 to about 25:75, with the ratio of lactic acid to glycolic acid of about 65:35 being more preferred.

Poly(lactic/glycolic acid) can be prepared by the procedures set forth in U.S. Pat. No. 4,293,539 (Ludwig et al.), which is incorporated herein by reference. In brief, Ludwig prepares the copolymer by condensation of lactic acid and glycolic acid in the presence of a readily removable polymerization catalyst (e.g., a strong ion-exchange resin such as Dowex HCR-W2-H). The amount of catalyst is not critical to the polymerization, but typically is from about 0.01 to about 20 parts by weight relative to the total weight of combined lactic acid and glycolic acid. The polymerization reaction can be conducted without solvents at a temperature from about 100° C. to about 250° C. for about 48 to about 96 hours, preferably under a reduced pressure to facilitate removal of water and by-products. Poly(lactic/glycolic acid) is then recovered by filtering the molten reaction mixture in an organic solvent, such as dichloromethane or acetone, and then filtering to remove the catalyst.

Suitable plasticizers, for example, acetyl triethyl citrate, acetyl tributyl citrate, triethyl citrate, diethyl phthalate, dibutyl phthalate, or dibutyl sebacate, also can be admixed with the polymer used to make the sequestering subunit. Additives, such as coloring agents, talc and/or magnesium stearate, and other additives also can be used in making the present inventive sequestering subunit.

In certain embodiments, additives may be included in the compositions to improve the sequestering characteristics of the sequestering subunit. As described below, the ratio of additives or components with respect to other additives or components may be modified to enhance or delay improve sequestration of the agent contained within the subunit. Various amounts of a functional additive (i.e., a charge-neutralizing additive) may be included to vary the release of an antagonist, particularly where a water-soluble core (i.e., a sugar sphere) is utilized. For instance, it has been determined that the inclusion of a low amount of charge-neutralizing additive relative to sequestering polymer on a weight-by-weight basis may cause decreased release of the antagonist.

In certain embodiments, a surfactant may serve as a charge-neutralizing additive. Such neutralization may in certain embodiments reduce the swelling of the sequestering polymer by hydration of positively charged groups contained therein. Surfactants (ionic or non-ionic) may also be used in preparing the sequestering subunit. It is preferred that the surfactant be ionic. Suitable exemplary agents include, for example, alkylaryl sulphonates, alcohol sulphates, sulphosuccinates, sulphosuccinamates, sarcosinates or taurates and others. Additional examples include but are not limited to ethoxylated castor oil, benzalkonium chloride, polyglycolyzed glycerides, acetylated monoglycerides, sorbitan fatty acid esters, poloxamers, polyoxyethylene fatty acid esters, polyoxyethylene derivatives, monoglycerides or ethoxylated derivatives thereof, diglycerides or polyoxyethylene derivatives thereof, sodium docusate, sodium lauryl sulfate, dioctyl sodium sulphosuccinate, sodium lauryl sarcosinate and sodium methyl cocoyl taurate, magnesium lauryl sulfate, triethanolamine, cetrimide, sucrose laurate and other sucrose esters, glucose (dextrose) esters, simethicone, ocoxynol, dioctyl sodiumsulfosuccinate, polyglycolyzed glycerides, sodiumdodecylbenzene sulfonate, dialkyl sodiumsulfosuccinate, fatty alcohols such as lauryl, cetyl, and steryl, glycerylesters, cholic acid or derivatives thereof, lecithins, and phospholipids. These agents are typically characterized as ionic (i.e., anionic or cationic) or nonionic. In certain embodiments described herein, an anionic surfactant such as sodium lauryl sulfate (SLS) is preferably used (U.S. Pat. No. 5,725,883; U.S. Pat. No. 7,201,920; EP 502642A1; Shokri, et al. Pharm. Sci. 2003. The effect of sodium lauryl sulphate on the release of diazepam from solid dispersions prepared by cogrinding technique. Wells, et al. Effect of Anionic Surfactants on the Release of Chlorpheniramine Maleate From an Inert, Heterogeneous Matrix. Drug Development and Industrial Pharmacy 18(2) (1992): 175-186. Rao, et al. “Effect of Sodium Lauryl Sulfate on the Release of Rifampicin from Guar Gum Matrix.” Indian Journal of Pharmaceutical Science (2000): 404-406; Knop, et al. Influence of surfactants of different charge and concentration on drug release from pellets coated with an aqueous dispersion of quaternary acrylic polymers. STP Pharma Sciences, Vol. 7, No. 6, (1997) 507-512). Other suitable agents are known in the art.

As shown herein, SLS is particularly useful in combination with Eudragit RS when the sequestering subunit is built upon a sugar sphere substrate. The inclusion of SLS at less than approximately 6.3% on a weight-to-weight basis relative to the sequestering polymer (i.e., Eudragit RS) may provide a charge neutralizing function (theoretically 20% and 41% neutralization, respectfully), and thereby significantly slow the release of the active agent encapsulated thereby (i.e., the antagonist naltrexone). Inclusion of more than approximately 6.3% SLS relative to the sequestering polymer appears to increase release of the antagonist from the sequestering subunit. With respect to SLS used in conjunction with Eudragit® RS, it is preferred that the SLS is present at approximately 1%, 2%, 3%, 4% or 5%, and typically less than 6% on a w/w basis relative to the sequestering polymer (i.e., Eudragit® RS). In preferred embodiments, SLS may be present at approximately 1.6% or approximately 3.3% relative to the sequestering polymer. As discussed above, many agents (i.e., surfactants) may substitute for SLS in the compositions disclosed herein.

Additionally useful agents include those that may physically block migration of the antagonist from the subunit and/or enhance the hydrophobicity of the barrier. One exemplary agent is talc, which is commonly used in pharmaceutical compositions (Pawar et al. Agglomeration of Ibuprofen With Talc by Novel Crystallo-Co-Agglomeration Technique. AAPS PharmSciTech. 2004; 5(4): article 55). As shown in the Examples, talc is especially useful where the sequestering subunit is built upon a sugar sphere core. Any form of talc may be used, so long as it does not detrimentally affect the function of the composition. Most talc results from the alteration of dolomite (CaMg(CO₃)₂ or magnesite (MgO) in the presence of excess dissolved silica (SiO₂) or by altering serpentine or quartzite. Talc may be include minerals such as tremolite (CaMg₃(SiO₃)₄), serpentine (3MgO.2SiO₂.2H₂O), anthophyllite (Mg₇.(OH)₂.(Si₄O₁₁)₂), magnesite, mica, chlorite, dolomite, the calcite form of calcium carbonate (CaCO₃), iron oxide, carbon, quartz, and/or manganese oxide. The presence of such impurities may be acceptable in the compositions described herein provided the function of the talc is maintained. It is preferred that that talc be USP grade. As mentioned above, the function of talc as described herein is to enhance the hydrophobicity and therefore the functionality of the sequestering polymer. Many substitutes for talc may be utilized in the compositions described herein as may be determined by one of skill in the art.

It has been determined that the ratio of talc to sequestering polymer may make a dramatic difference in the functionality of the compositions described herein. For instance, the Examples described below demonstrate that the talc to sequestering polymer ratio (w/w) is important with respect to compositions designed to prevent the release of naltrexone therefrom. It is shown therein that inclusion of an approximately equivalent amount (on a weight-by-weight basis) of talc and Eudragits® RS results in a very low naltrexone release profile. In contrast, significantly lower or higher both a lower (69% w/w) and a higher (151% w/w) talc:Eudragit® RS ratios result in increased release of naltrexone release. Thus, where talc and Eudragit® RS are utilized, it is preferred that talc is present at approximately any of 75%, 80%, 85%, 90%, 95%, 100%, 105%, 110%, 115%, 120%, 125%, 142%, or 150% w/w relative to Eudragit® RS. As described above, the most beneficial ratio for other additives or components will vary and may be determined using standard experimental procedures.

In certain embodiments, such as where a water-soluble core is utilized, it may be useful to include agents that may affect the osmotic pressure of the composition (i.e., an osmotic pressure regulating agent) (see, in general, WO 2005/046561A2 and WO 2005/046649 A2 relating to Eudramode®). The use of an osmotic pressure regulating agent may depend on the choice of agonist or antagonists and the forms (salts) of agonist and antagonist chosen. To the extent that an osmotic pressure regulating agent is chose for a particular composition, the agent is preferably applied to the Eudragit® RS/talc layer described above. In a pharmaceutical unit comprising a sequestering subunit overlayed by an active agent (i.e., a controlled-release agonist preparation), the osmotic pressure regulating agent is preferably positioned immediately beneath the active agent layer. Suitable osmotic pressure regulating agents may include, for instance, hydroxypropylmethyl cellulose (HPMC) or chloride ions (i.e., from NaCl), or a combination of HPMC and chloride ions (i.e., from NaCl). Other ions that may be useful include bromide or iodide. The combination of sodium chloride and HPMC may be prepared in water or in a mixture of ethanol and water, for instance. HPMC is commonly utilized in pharmaceutical compositions (see, for example, U.S. Pat. Nos. 7,226,620 and 7,229,982). In certain embodiments, HPMC may have a molecular weight ranging from about 10,000 to about 1,500,000, and typically from about 5000 to about 10,000 (low molecular weight HPMC). The specific gravity of HPMC is typically from about 1.19 to about 1.31, with an average specific gravity of about 1.26 and a viscosity of about 3600 to 5600. HPMC may be a water-soluble synthetic polymer. Examples of suitable, commercially available hydroxypropyl methylcellulose polymers include Methocel K100 LV and Methocel K4M (Dow). Other HPMC additives are known in the art and may be suitable in preparing the compositions described herein. In certain embodiments, it is preferred that the charge-neutralizing additive (i.e., NaCl) is included at less than approximately 1, 2, 3, 4, 5, 6, 7, 8, 9, or 10% of the composition on a weight-by-weight basis. In other preferred embodiments, the charge-neutralizing additive is present at approximately 4% of the composition on a weight-by-weight basis.

Thus, in one embodiment, a sequestering subunit built upon a sugar sphere substrate is provided comprising a sequestering polymer (i.e., Eudragit® RS) in combination with several optimizing agents, including sodium lauryl sulfate (SLS) as a charge-neutralizing agent to reduce swelling of the film by hydration of the positively charged groups on the polymer; talc to create a solid impermeable obstacle to naltrexone transport through the film and as a hydrophobicity-enhancing agent; and a chloride ion (i.e., as NaCl) as an osmotic pressure reducing agent. The ratio of each of the additional ingredients relative to the sequestering polymer was surprisingly found to be important to the function of the sequestering subunit. For instance, the Examples provide a sequestering subunit including a sequestering polymer and the optimizing agents SLS at less than 6%, preferably 1-4%, and even more preferably 1.6% or 3.3% on a w/w basis relative to Eudragit RS; talc in an amount approximately equal to Eudragit® RS (on a w/w basis); and, NaCl present at approximately 4% on a w/w basis.

The therapeutic agent can be an opioid agonist. By “opioid” is meant to include a drug, hormone, or other chemical or biological substance, natural or synthetic, having a sedative, narcotic, or otherwise similar effect(s) to those containing opium or its natural or synthetic derivatives. By “opioid agonist,” sometimes used herein interchangeably with terms “opioid” and “opioid analgesic,” is meant to include one or more opioid agonists, either alone or in combination, and is further meant to include the base of the opioid, mixed or combined agonist-antagonists, partial agonists, pharmaceutically acceptable salts thereof, stereoisomers thereof, ethers thereof, esters thereof, and combinations thereof.

Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. In a preferred embodiment, the opioid agonist comprises oxycodone or hydrocodone and is present in the dosage form in an amount of about 15 to about 45 mg, and the opioid antagonist comprises naltrexone and is present in the dosage form in an amount of about 0.5 to about 5 mg.

Pharmaceutically acceptable salts of the antagonist or agonist agents discussed herein include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N′-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like.

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms described herein may include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use as described herein, and processes for their manufacture: for example, U.S. Pat. Nos. 5,266,331; 5,549,912; 5,508,042; and 5,656.295, which are incorporated herein by reference. The opioid agonist can comprise tramadol and the sustained-release oral dosage forms can include from about 25 mg to 800 mg tramadol per dosage unit.

Pharmaceutically acceptable salts of the antagonist or agonist agents discussed herein include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N′-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like.

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms may include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use as described herein, and processes for their manufacture: for example, U.S. Pat. Nos. 5,266,331; 5,549,912; 5,508,042; and 5,656,295, which are incorporated herein by reference. The opioid agonist can comprise tramadol and the sustained-release oral dosage forms can include from about 25 mg to 800 mg tramadol per dosage unit.

In a preferred embodiment, the oral dosage form may be formulated to provide for an increased duration of therapeutic action allowing once-daily dosing. In general, a release-retarding material is used to provide the increased duration of therapeutic action. Preferably, once-daily dosing is provided by the dosage forms. In certain embodiments the blood level of the agonist reaches its maximum concentration (Tₘₐₓ) about 8 to 24 hours after administration. In preferred embodiments Tₘₐₓ is reached about 10 to about 16 hours after administration. In certain embodiments the ratio of C₂₄ (the concentration of the agonist in the blood at 24 hours) to Cₘₐₓ (the maximum concentration of the agonist in the blood) is between about 0.2 and 0.8.

Preferred release-retarding materials include acrylic polymers, alkylcelluloses, shellac, zein, hydrogenated vegetable oil, hydrogenated castor oil, and combinations thereof. In certain preferred embodiments, the release-retarding material is a pharmaceutically acceptable acrylic polymer, including acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, and glycidyl methacrylate copolymers. In certain preferred embodiments, the acrylic polymer comprises one or more ammonio methacrylate copolymers. Ammonio methacrylate copolymers are well-known in the art, and are described in NF21, the 21^{st} edition of the National Formulary, published by the United States Pharmacopeial Convention Inc. (Rockville, Md.), as fully polymerized copolymers of acrylic and methacrylic acid esters with a low content of quaternary ammonium groups. In other preferred embodiments, the release-retarding material is an alkyl cellulosic material, such as ethylcellulose. Those skilled in the art will appreciate that other cellulosic polymers, including other alkyl cellulosic polymers, can be substituted for part or all of the ethylcellulose.

Release-modifying agents, which affect the release properties of the release-retarding material, also can be used. In a preferred embodiment, the release-modifying agent functions as a pore-former. The pore-former can be organic or inorganic, and include materials that can be dissolved, extracted or leached from the coating in the environment of use. The pore-former can comprise one or more hydrophilic polymers, such as hydroxypropylmethylcellulose. In certain preferred embodiments, the release-modifying agent is selected from hydroxypropylmethylcellulose, lactose, metal stearates, and combinations thereof.

The release-retarding material can also include an erosion-promoting agent, such as starch and gums; a release-modifying agent useful for making microporous lamina in the environment of use, such as polycarbonates comprised of linear polyesters of carbonic acid in which carbonate groups reoccur in the polymer chain; and/or a semi-permeable polymer.

With respect to the present inventive compositions, the composition is preferably an oral dosage form. By “oral dosage form” is meant to include a unit dosage form prescribed or intended for oral administration comprising subunits. Desirably, the composition comprises the sequestering subunit coated with the therapeutic agent in releasable form, thereby forming a composite subunit comprising the sequestering subunit and the therapeutic agent. Accordingly, this disclosure further provides a capsule suitable for oral administration comprising a plurality of such composite subunits.

Alternatively, the oral dosage form can comprise any of the sequestering subunits disclosed herein in combination with a therapeutic agent subunit, wherein the therapeutic agent subunit comprises the therapeutic agent in releasable form. In this respect, a capsule suitable for oral administration comprising a plurality of sequestering subunits of the invention and a plurality of therapeutic subunits, each of which comprises a therapeutic agent in releasable form is provided. With respect to the presently disclosed compositions, the composition may preferably be an oral dosage form. By “oral dosage form” is meant to include a unit dosage form prescribed or intended for oral administration comprising subunits. Desirably, the composition comprises the sequestering subunit coated with the therapeutic agent in releasable form, thereby forming a composite subunit comprising the sequestering subunit and the therapeutic agent. Accordingly, a capsule suitable for oral administration comprising a plurality of such composite subunits is also provided.

Alternatively, the oral dosage form can comprise any of the sequestering subunits in combination with a therapeutic agent subunit, wherein the therapeutic agent subunit comprises the therapeutic agent in releasable form. In this respect, a capsule suitable for oral administration comprising a plurality of sequestering subunits of the invention and a plurality of therapeutic subunits, each of which comprises a therapeutic agent in releasable form is provided.

When the blocking agent is a system comprising a first antagonist-impermeable material and a core, the sequestering subunit can be in one of several different forms. For example, the system can further comprise a second antagonist-impermeable material, in which case the sequestering unit comprises an antagonist, a first antagonist-impermeable material, a second antagonist-impermeable material, and a core. In this instance, the core is coated with the first antagonist-impermeable material, which, in turn, is coated with the antagonist, which, in turn, is coated with the second antagonist-impermeable material. The first antagonist-impermeable material and second antagonist-impermeable material substantially prevent release of the antagonist from the sequestering subunit in the gastrointestinal tract for a time period that is greater than 24 hours. In some instances, it is preferable that the first antagonist-impermeable material is the same as the second antagonist-impermeable material. In other instances, the first antagonist-impermeable material is different from the second antagonist-impermeable material. It is within the skill of the ordinary artisan to determine whether or not the first and second antagonist-impermeable materials should be the same or different. Factors that influence the decision as to whether the first and second antagonist-impermeable materials should be the same or different can include whether a layer to be placed over the antagonist-impermeable material requires certain properties to prevent dissolving part or all of the antagonist-impermeable layer when applying the next layer or properties to promote adhesion of a layer to be applied over the antagonist-impermeable layer.

Alternatively, the antagonist can be incorporated into the core, and the core is coated with the first antagonist-impermeable material. In this case, a sequestering subunit comprising an antagonist, a core and a first antagonist-impermeable material, wherein the antagonist is incorporated into the core and the core is coated with the first antagonist-impermeable material, and wherein the first antagonist-impermeable material substantially prevents release of the antagonist from the sequestering subunit in the gastrointestinal tract for a time period that is greater than 24 hours may be provided. By “incorporate” and words stemming therefrom, as used herein is meant to include any means of incorporation, e.g., homogeneous dispersion of the antagonist throughout the core, a single layer of the antagonist coated on top of a core, or a multi-layer system of the antagonist, which comprises the core.

In another alternative embodiment, the core comprises a water-insoluble material, and the core is coated with the antagonist, which, in turn, is coated with the first antagonist-impermeable material. In this case, a sequestering subunit comprising an antagonist, a first antagonist-impermeable material, and a core, which comprises a water-insoluble material, wherein the core is coated with the antagonist, which, in turn, is coated with the first antagonist-impermeable material, and wherein the first antagonist-impermeable material substantially prevents release of the antagonist from the sequestering subunit in the gastrointestinal tract for a time period that is greater than 24 hours is provided. The term “water-insoluble material” as used herein means any material that is substantially water-insoluble. The term “substantially water-insoluble” does not necessarily refer to complete or 100% water-insolubility. Rather, there are varying degrees of water insolubility of which one of ordinary skill in the art recognizes as having a potential benefit. Preferred water-insoluble materials include, for example, microcrystalline cellulose, a calcium salt, and a wax. Calcium salts include, but are not limited to, a calcium phosphate (e.g., hydroxyapatite, apatite; etc.), calcium carbonate, calcium sulfate, calcium stearate, and the like. Waxes include, for example, carnuba wax, beeswax, petroleum wax, candelilla wax, and the like.

In one embodiment, the sequestering subunit includes an antagonist and a seal coat where the seal coat forms a layer physically separating the antagonist within the sequestering subunit from the agonist which is layered upon the sequestering subunit. In one embodiment, the seal coat comprises one or more of an osmotic pressure regulating agent, a charge-neutralizing additive, a sequestering polymer hydrophobicity-enhancing additive, and a first sequestering polymer (each having been described above). In such embodiments, it is preferred that the osmotic pressure regulating agent, charge-neutralizing additive, and/or sequestering polymer hydrophobicity-enhancing additive, respectively where present, are present in proportion to the first sequestering polymer such that no more than 10% of the antagonist is released from the intact dosage form. Where an opioid antagonist is used in the sequestering subunit and the intact dosage form includes an opioid agonist, it is preferred that ratio of the osmotic pressure regulating agent, charge-neutralizing additive, and/or sequestering polymer hydrophobicity-enhancing additive, respectively where present, in relation to the first sequestering polymer is such that the physiological effect of the opioid agonist is not diminished when the composition is in its intact dosage form or during the normal course digestion in the patient. Release may be determined as described above using the USP paddle method (optionally using a buffer containing a surfactant such as Triton X-100) or measured from plasma after administration to a patient in the fed or non-fed state. In one embodiment, plasma naltrexone levels are determined; in others, plasma 6-beta naltrexol levels are determined. Standard tests may be utilized to ascertain the antagonist's effect on agonist function (i.e., reduction of pain).

When the blocking agent is a system comprising a first antagonist-impermeable material and a core, the sequestering subunit can be in one of several different forms. For example, the system can further comprise a second antagonist-impermeable material, in which case the sequestering unit comprises an antagonist, a first antagonist-impermeable material, a second antagonist-impermeable material, and a core. In this instance, the core is coated with the first antagonist-impermeable material, which, in turn, is coated with the antagonist, which, in turn, is coated with the second antagonist-impermeable material. The first antagonist-impermeable material and second antagonist-impermeable material substantially prevent release of the antagonist from the sequestering subunit in the gastrointestinal tract for a time period that is greater than 24 hours. In some instances, it is preferable that the first antagonist-impermeable material is the same as the second antagonist-impermeable material. In other instances, the first antagonist-impermeable material is different from the second antagonist-impermeable material. It is within the skill of the ordinary artisan to determine whether or not the first and second antagonist-impermeable materials should be the same or different. Factors that influence the decision as to whether the first and second antagonist-impermeable materials should be the same or different can include whether a layer to be placed over the antagonist-impermeable material requires certain properties to prevent dissolving part or all of the antagonist-impermeable layer when applying the next layer or properties to promote adhesion of a layer to be applied over the antagonist-impermeable layer.

Alternatively, the antagonist can be incorporated into the core, and the core is coated with the first antagonist-impermeable material. In this case, a sequestering subunit comprising an antagonist, a core and a first antagonist-impermeable material, wherein the antagonist is incorporated into the core and the core is coated with the first antagonist-impermeable material, and wherein the first antagonist-impermeable material substantially prevents release of the antagonist from the sequestering subunit in the gastrointestinal tract for a time period that is greater than 24 hours is provided. By “incorporate” and words stemming therefrom, as used herein is meant to include any means of incorporation, e.g., homogeneous dispersion of the antagonist throughout the core, a single layer of the antagonist coated on top of a core, or a multi-layer system of the antagonist, which comprises the core.

In another alternative embodiment, the core comprises a water-insoluble material, and the core is coated with the antagonist, which, in turn, is coated with the first antagonist-impermeable material. In this case, a sequestering subunit comprising an antagonist, a first antagonist-impermeable material, and a core, which comprises a water-insoluble material, wherein the core is coated with the antagonist, which, in turn, is coated with the first antagonist-impermeable material, and wherein the first antagonist-impermeable material substantially prevents release of the antagonist from the sequestering subunit in the gastrointestinal tract for a time period that is greater than 24 hours is provided. The term “water-insoluble material” as used herein means any material that is substantially water-insoluble. The term “substantially water-insoluble” does not necessarily refer to complete or 100% water-insolubility. Rather, there are varying degrees of water insolubility of which one of ordinary skill in the art recognizes as having a potential benefit. Preferred water-insoluble materials include, for example, microcrystalline cellulose, a calcium salt, and a wax. Calcium salts include, but are not limited to, a calcium phosphate (e.g., hydroxyapatite, apatite; etc.), calcium carbonate, calcium sulfate, calcium stearate, and the like. Waxes include, for example, carnuba wax, beeswax, petroleum wax, candelilla wax, and the like.

In one embodiment, the sequestering subunit includes an antagonist and a seal coat where the seal coat forms a layer physically separating the antagonist within the sequestering subunit from the agonist which is layered upon the sequestering subunit. In one embodiment, the seal coat comprises one or more of an osmotic pressure regulating agent, a charge-neutralizing additive, a sequestering polymer hydrophobicity-enhancing additive, and a first sequestering polymer (each having been described above). In such embodiments, it is preferred that the osmotic pressure regulating agent, charge-neutralizing additive, and/or sequestering polymer hydrophobicity-enhancing additive, respectively where present, are present in proportion to the first sequestering polymer such that no more than 10% of the antagonist is released from the intact dosage form. Where an opioid antagonist is used in the sequestering subunit and the intact dosage form includes an opioid agonist, it is preferred that ratio of the osmotic pressure regulating agent, charge-neutralizing additive, and/or sequestering polymer hydrophobicity-enhancing additive, respectively where present, in relation to the first sequestering polymer is such that the physiological effect of the opioid agonist is not diminished when the composition is in its intact dosage form or during the normal course digestion in the patient. Release may be determined as described above using the USP paddle method (optionally using a buffer containing a surfactant such as Triton X-100) or measured from plasma after administration to a patient in the fed or non-fed state. In one embodiment, plasma naltrexone levels are determined; in others, plasma 6-beta naltrexol levels are determined. Standard tests may be utilized to ascertain the antagonist's effect on agonist function (i.e., reduction of pain).

In certain embodiments, the release of the antagonist of the sequestering subunit or composition is expressed in terms of a ratio of the release achieved after tampering, e.g., by crushing or chewing, relative to the amount released from the intact formulation. The ratio is, therefore, expressed as [Crushed]:[Whole], and it is desired that this ratio have a numerical range of at least about 4:1 or greater (e.g., crushed release within 1 hour/intact release in 24 hours). In certain embodiments, the ratio of the therapeutic agent and the antagonist, present in the sequestering subunit, is about 1:1 to about 50:1 by weight, preferably about 1:1 to about 20:1 by weight or 15:1 to about 30:1 by weight. The weight ratio of the therapeutic agent to antagonist refers to the weight of the active ingredients. Thus, for example, the weight of the therapeutic agent excludes the weight of the coating, matrix, or other component that renders the antagonist sequestered, or other possible excipients associated with the antagonist particles. In certain preferred embodiments, the ratio is about 1:1 to about 10:1 by weight. Because in certain embodiments the antagonist is in a sequestered from, the amount of such antagonist within the dosage form can be varied more widely than the therapeutic agent/antagonist combination dosage forms, where both are available for release upon administration, as the formulation does not depend on differential metabolism or hepatic clearance for proper functioning. For safety reasons, the amount of the antagonist present in a substantially non-releasable form is selected as not to be harmful to humans, even if fully released under conditions of tampering.

Compositions comprising a plurality of multi-layer pellets comprising a water soluble core; an antagonist containing layer comprising naltrexone HCl coating the core; a sequestering polymer layer coating the antagonist containing layer; an agonist layer comprising an opioid agonist coating the sequestering polymer layer; and a controlled release layer coating the agonist layer, wherein the weight of the naltrexone HCl comprises at least 5% the combined weight of the water soluble core, antagonist layer and sequestering polymer layer and wherein the agonist is substantially released and the naltrexone HCl is substantially sequestered upon administration to a human being are provided. In certain embodiments the naltrexone HCl comprises from about 5% to about 30% of the combined weight of the water soluble core, antagonist layer and sequestering polymer layer. In other embodiments the naltrexone HCl comprises from about 5% to about 20% of the combined weight of the water soluble core, antagonist layer and sequestering polymer layer. In preferred embodiments the naltrexone HCl comprises from about 5% to about 10% of the combined weight of the water soluble core, antagonist layer and sequestering polymer layer. In other preferred embodiments the naltrexone HCl comprises from about 6% to about 10%, or from about 7% to about 10% or about 8% to about 10% of the combined weight of the water soluble core, antagonist layer and sequestering polymer layer.

The compositions of the invention are particularly well-suited for use in preventing abuse of a therapeutic agent. In this regard, a method of preventing abuse of a therapeutic agent by a human being is provided. The method comprises incorporating the therapeutic agent into any of the compositions described herein. Upon administration of the composition described herein to the person, the antagonist is substantially prevented from being released in the gastrointestinal tract for a time period that is greater than 24 hours. However, if a person tampers with the compositions, the sequestering subunit, which is mechanically fragile, will break and thereby allow the antagonist to be released. Since the mechanical fragility of the sequestering subunit is the same as the therapeutic agent in releasable form, the antagonist will be mixed with the therapeutic agent, such that separation between the two components is virtually impossible.

The effectiveness of treatment of chronic moderate to severe pain (focusing on osteoarthritis of the hip or knee) is typically measured by mean change in diary Brief Pain Inventory (BPI) score of average pain (daily scores of average pain averaged over 7 days; in-clinic BPI and/or daily diary BPI (worst, least, and current pain)), WOMAC Osteoarthritis Index, Medical Outcomes Study (MOS) Sleep Scale, Beck Depression Inventory, and Patient Global Impression of Change (PGIC). The safety and tolerability of opioid medications such as Kadian NT are compared to placebo using Adverse Events (AEs), clinical laboratory data, vital signs, and two measures of opioid withdrawal: Subjective Opiate Withdrawal Scale (SOWS) and Clinical Opiate Withdrawal Scale (COWS).

The compositions described herein may comprise a plurality of multi-layer pellets comprising a water soluble core; an antagonist containing layer comprising naltrexone HCl coating the core; a sequestering polymer layer coating the antagonist containing layer; an agonist layer comprising an opioid agonist coating the sequestering polymer layer; and a controlled release layer coating the agonist layer; wherein the weight of the naltrexone HCl comprises at least 5% the combined weight of the water soluble core, antagonist layer and sequestering polymer layer and wherein the agonist is substantially released and the naltrexone HCl is substantially sequestered upon administration to a human being.

All references cited in this disclosure are incorporated herein in their entirety. The following non-limiting examples describe particular embodiments of the compositions and methods described herein.

### EXAMPLES

### Example 1

### 20% Oxycodone Formulation

### Sieved Sugar Spheres

Prior to seal coating, the sugar spheres are sieved to remove undersize spheres. The acceptably sized sugar spheres are collected and used in the seal coating process.

### Seal-Coated Sugar Spheres

### Charge

600 to 710 μm mesh sugar spheres

| Seal Coating dispersion | Solution | Solids | Apply |
|---|---|---|---|
| SD3A Ethanol | 80.00% | — | 4532.8 g |
| Dibutyl sebacate NF | 0.50% | 2.50% | 28.3 g |
| Ethylcellulose 50 NF | 5.00% | 25.00% | 283.3 g |
| Magnesium stearate | 2.00% | 10.00% | 113.3 g |
| Talc USP | 12.50% | 62.50% | 708.3 g |
| Total | 100.00% | 100.00% | 5666.0 g |

The manufacturing of seal-coated sugar spheres involves preparation of the seal coating dispersion and spray coating of the dispersion onto the sieved sugar spheres.

The seal coating dispersion is prepared by first dissolving dibutyl sebacate and ethylcellulose in alcohol. Talc and magnesium stearate are then added and dispersed uniformly into the solution prior to the seal coating operation. Mixing is continued until all the dispersion is applied.

The seal coating dispersion is sprayed onto the sieved sugar spheres utilizing a Wurster insert in the fluid bed. The coat application is performed under pre-defined process parameter settings. After all the seal coating dispersion has been sprayed, alcohol is sprayed on the product to flush the pump lines and spray nozzles. Once the flushing is complete, the product spheres are dried, discharged, weighed and sieved. Oversized and undersized spheres are subsequently discarded. The acceptably sized spheres are further processed into the next step.

### Naltrexone Hydrochloride Pellets Overview

Manufacturing of naltrexone hydrochloride pellets starts with naltrexone hydrochloride (NT) drug layering onto the seal-coated sugar spheres to form naltrexone cores (NT drug layering represents approximately 18.5% total weight gain). Subsequently, these naltrexone cores undergo a two-step coating of the sequestering membrane (also called barrier coat), which represents approximately 122.6% total weight gain. All drug layering and coat applications are performed in a fluid bed equipped with a Wurster insert. Curing is performed in an oven after each step of the barrier coating and sieving is conducted on the final cured finished pellets.

### NT Cores

### Charge

Seal Coated Sugar Spheres (−18/+30 mesh): 1700 g

| Naltrexone HCl Dispersion | Solutions | Solid | Apply |
|---|---|---|---|
| SD3A Ethanol | 63.07% | — | 956.7 g |
| Purified Water USP | 16.22% | — | 246 g |
| Ascorbic Acid USP | 1.16% | 5.60% | 17.6 g |
| HPC NF (75-150 cps) | 2.24% | 10.82% | 34.0 g |
| Naltrexone HCl USP | 11.81% | 57.02% | 179.2 g |
| Talc USP | 5.50% | 26.57% | 83.5 g |
| Total | 100.00% | 100.00% | 1517.0 g |

### Naltrexone Cores

The naltrexone dispersion is first prepared by dissolving ascorbic acid and hydroxypropyl cellulose into alcohol and purified water. Naltrexone hydrochloride and talc are then added and dispersed uniformly into the solution. Mixing is continued until all the dispersion is applied.

The naltrexone dispersion is sprayed onto the seal-coated sugar spheres utilizing a Wurster insert in the fluid bed. The drug coat application is performed under pre-defined process parameter settings. After all the naltrexone dispersion has been sprayed, alcohol is sprayed on the product to flush the pump lines and spray nozzles. Once the flushing is complete, the product cores are dried and discharged.

### NT Intermediate Pellets

Naltrexone HCl Cores (−18/+30 mesh): 1700 g

| Intermediate dispersion | Solution | Solids | Apply |
|---|---|---|---|
| SD3A Ethanol | 62.34% | — | 3249.8 g |
| Purified Water USP | 17.67% | — | 921 g |
| SLS NF | 0.64% | 3.20% | 33.4 g |
| Dibutyl sebacate NF | 0.96% | 4.79% | 49.9 g |
| Eudragit RS | 7.59% | 37.99% | 395.9 g |
| Talc USP | 10.80% | 54.02% | 563.0 g |
| Talc USP (dusting) | — | — | 11.6 g |
| Total | 100.00% | 100.00% | 5213.0 g |

### NT Finished Pellets

### Charge

Naltrexone HCl Intermediate Pellets (−16/+25 mesh): 1700.0 g

| Intermediate dispersion | Solution | Solids | Apply |
|---|---|---|---|
| SD3A Ethanol | 62.34% | — | 3249.8 g |
| Purified Water USP | 17.67% | — | 921 g |
| SLS NF | 0.64% | 3.20% | 33.4 g |
| Dibutyl sebacate NF | 0.96% | 4.79% | 49.9 g |
| Eudragit RS | 7.59% | 37.99% | 395.9 g |
| Talc USP | 10.80% | 54.02% | 563.0 g |
| Talc USP (dusting) | — | — | 11.6 g |
| Total | 100.00% | 100.00% | 5213.0 g |

### Naltrexone Pellets (Intermediate and Finished)

The barrier coating process is performed in two steps—the first step produces naltrexone intermediate pellets (61.3% weight gain based on naltrexone cores) and the second step produces finished pellets (total of 122.6% weight gain based on naltrexone cores).

The barrier coating dispersions for both the intermediate pellets and finished pellets are prepared in the same way. Sodium lauryl sulfate, dibutyl sebacate, ammonio methacrylate copolymer type B (Eudragit® RS) are first dissolved into alcohol and purified water. Talc is dispersed into the solution before barrier coating commences. Mixing is continued until all the dispersion is applied.

For the naltrexone intermediate pellets, the barrier coating dispersion is sprayed onto naltrexone cores utilizing a Wurster insert in the fluid bed. The coat application is performed under pre-defined process parameter settings. After all the barrier coating dispersion has been sprayed, alcohol is sprayed onto the product to flush the pump lines and spray nozzles. Once the flushing is complete, the product pellets are dried and dusted with talc. The intermediate pellets are then transferred onto oven trays for curing. After curing, the intermediate pellets are weighed and sieved. The oversized and undersized pellets are subsequently discarded. The acceptably sized naltrexone intermediate pellets are further processed to the finished naltrexone pellets.

For the finished naltrexone pellets, the barrier coating dispersion is sprayed onto the cured naltrexone intermediate pellets utilizing a Wurster insert in the fluid bed. The same procedures (spraying, alcohol flushing, drying, dusting, curing and sieving) as the intermediate pellets are followed. The oversized and undersized pellets are subsequently discarded. The acceptably sized finished naltrexone pellets are further processed to the next step.

### ALO-02 Cores

### Charge

### Naltrexone HCl Pellets (−14/+25 Mesh): 2000 g

| Oxycodone HCl Dispersion | Solutions | Solids | Apply |
|---|---|---|---|
| SD3A Ethanol | 80.35% | — | 2408.7 g |
| HPC NF (75-150 cps) | 2.69% | 13.70% | 80.7 g |
| Oxycodone HCl USP | 11.31% | 57.54% | 339.0 g |
| Talc USP | 5.65% | 28.77% | 169.5 g |

Oxycodone Hydrockloride Cores with Sequestered Naltrexone Hydrochloride

The manufacturing of oxycodone hydrochloride cores with sequestered naltrexone hydrochloride involves preparation of the oxycodone hydrochloride drug dispersion and spray coating of the dispersion onto naltrexone hydrochloride pellets.

The oxycodone hydrochloride drug dispersion is prepared by first dissolving hydroxypropyl cellulose into alcohol. Oxycodone hydrochloride is added and uniformly dispersed into the solution prior to drug layering. Mixing is continued until all the dispersion is applied.

The oxycodone hydrochloride drug dispersion is sprayed onto naltrexone hydrochloride pellets utilizing a Wurster insert in the fluid bed. The drug layer application is performed under pre-defined process parameter settings. After all the drug dispersion has been sprayed, alcohol is sprayed on the product to flush the pump lines and spray nozzles. Once the flushing is complete, the product pellets are dried and discharged. The cores are then weighed and sieved. The oversized and undersized cores are rejected. The final acceptably sized cores are further processed to the next step.

### ALO-02 Pellets

### Charge

### Oxycodone HCl Cores: 2250.0 g

| Top Coat | | | |
|---|---|---|---|
| Dispersion | Solution | Solids | Apply |
| SD3A Ethanol | 85.71% | — | 2762.3 g |
| Diethyl phthalate | 1.05% | 7.34% | 33.8 g |
| NF | | | |
| PEG 6000 | 1.84% | 12.85% | 59.2 g |
| Eudragit L100-55 | 0.74% | 5.19% | 23.9 g |
| Ethylcellulose 50 | 5.90% | 41.24% | 190.0 g |
| NF | | | |
| Talc USP | 4.77% | 33.38% | 153.8 g |
| Talc USP (dusting) | — | — | 11.6 g |
| Total | 100.00% | 100.00% | 3223.0 g |

Final Pellets—Oxycodone Hydrochloride Extended Release with Sequestered Naltrexone Hydrochloride

The manufacturing of oxycodone hydrochloride extended-release with sequestered naltrexone hydrochloride pellets involves preparation of the coating dispersion and spray coating of the dispersion onto oxycodone hydrochloride cores with sequestered naltrexone.

The coating dispersion is prepared by first dissolving diethyl phthalate, polyethylene glycol (PEG), methacrylic acid copolymer type C (Eudragit® L100-55), and ethylcellulose into alcohol. Talc is then added and uniformly dispersed into the coating solution. Mixing is continued until all the dispersion is completely sprayed.

Utilizing a Wurster insert in the fluid bed, the coating dispersion is sprayed onto oxycodone hydrochloride cores with sequestered naltrexone. The coat application is performed under pre-defined process parameter settings. After all the coating dispersion has been sprayed, alcohol is sprayed on the product to flush the pump lines and spray nozzles. Once the flushing is complete, the product pellets are dried and dusted with talc. The pellets are then weighed and sieved. The oversized and undersized pellets are rejected. The final acceptably sized pellets are further processed to the next step.

### Oxycodone Hydrockloride and Naltrexone Hydrochloride Extended Release Capsules

Target fill weight for individual capsule is calculated based on the fractional potency of oxycodone hydrochloride for the final pellets and the capsule strength. Acceptable weight limits are calculated and must be between ±5% of the target fill weight. Designated capsule shells and pellets are dispensed. The capsules are filled with pellets either manually or by an automated encapsulation machine.

The overall amount of each component by weight and percentage in a batch production as well as the weight of each component per capsule is presented in the table below:

Composition of Oxycodone Hydrochloride and Naltrexone Hydrochloride
Extended Release Capsules, 40 mg/8 mg

| | Quality | | | |
|---|---|---|---|---|
| | Standard | | | |
| Component | Reference | % w/w | mg/capsule | Function |
| Active Components: | | | | |
| Oxycodone hydrochloride | USP | 10.82 | 40.04¹ | Active component |
| Naltrexone hydrochloride | USP | 2.16 | 8.01¹ | Active component² |
| Inactive Components: | | | | |
| Talc (1656) | USP | 39.17 | 144.94 | Hydrophobic glidant |
| | | | | Anti-sticking agent |
| | | | | Glidant |
| | | | | Anti-static agent |
| Ammonio methacrylic | NF | 14.84 | 54.92 | Sequestering membrane |
| copolymer (Type B | | | | polymer component |
| Powder) | | | | |
| (Eudragit ® RS PO) | | | | |
| Sugar spheres | NF | 12.32 | 45.59 | Seed core |
| (25-30 mesh) | | | | |
| Ethylcellulose | NF | 9.03 | 33.42 | Rate controlling polymer |
| (50 cps) | | | | component |
| | | | | Film coating agent |
| Hydroxypropyl cellulose | NF | 2.99 | 11.05 | Film coating agent |
| (75-150 cps) | | | | |
| (Klucel ® LF) | | | | |
| Polyethylene glycol | NF | 2.17 | 8.05 | Rate controlling polymer |
| (6000) | | | | component |
| Dibutyl sebacate | NF | 2.08 | 7.68 | Plasticizer |
| Sodium lauryl sulfate | NF | 1.25 | 4.63 | Sequestering membrane |
| | | | | component |
| Diethyl phthalate | NF | 1.24 | 4.59 | Plasticizer |
| Methacrylic acid copolymer | NF | 0.88 | 3.25 | Rate controlling polymer |
| (Type C Powder) | | | | component |
| (Eudragit ® L100-55) | | | | |
| Magnesium stearate | NF | 0.82 | 3.04 | Hydrophobic agent |
| (vegetable sourced) | | | | |
| Ascorbic acid | USP | 0.21 | 0.79 | pH controlling agent |
| Ethanol (Denatured SD3A) | In-house | — | — | Solvent³ |
| Purified water | USP | — | — | Solvent³ |
| Hard gelatin capsule shell | In-house | — | (1 each) | Capsule component |
| (Size #0, Pink Opaque)⁴ | | | | |
| Total Capsule Fill Weight | | 100% | 370 | |

¹Amount charged to batch may be corrected for potency and/or moisture.
²Although naltrexone hydrochloride is an active component, the formulation is designed to sequester the naltrexone hydrochloride so that it is not released.
³Processing agent used in manufacturing

### Example 2

### Oxycodone Dissolution Profile for Oxycodone 20%

Six sample capsules of oxycodone/naltrexone beads, manufactured as described in Example 1, were tested for in vitro dissolution by placing the capsules. The results are shown in the table below:

| | | Oxycodone | Total oxycodone | % Oxycodone |
|---|---|---|---|---|
| Hour | Vessel | released (mg) | released (mg) | released |
| 1 | 1 | 0.2320 | 0.2320 | 0.6 |
| | 2 | 0.3596 | 0.3596 | 1.9 |
| | 3 | 0.2295 | 0.2295 | 0.6 |
| | 4 | 0.3978 | 0.3978 | 1.0 |
| | 5 | 0.2110 | 0.2110 | 0.5 |
| | 6 | 0.2389 | 0.2389 | 0.6 |
| 2 | 1 | 1.1398 | 1.3718 | 3.4 |
| | 2 | 1.1013 | 1.4069 | 3.7 |
| | 3 | 1.1134 | 1.3429 | 3.4 |
| | 4 | 1.0755 | 1.4733 | 3.7 |
| | 5 | 1.0331 | 1.2441 | 3.1 |
| | 6 | 1.1369 | 1.3758 | 3.4 |
| 4 | 1 | 5.5251 | 5.7662 | 14.4 |
| | 2 | 5.2429 | 5.6113 | 14.0 |
| | 3 | 5.4402 | 5.6786 | 14.2 |
| | 4 | 5.1643 | 5.5707 | 13.9 |
| | 5 | 5.2464 | 5.4656 | 13.7 |
| | 6 | 5.6118 | 5.8598 | 14.6 |
| 8 | 1 | 16.8108 | 17.0963 | 42.7 |
| | 2 | 16.1140 | 16.5247 | 41.3 |
| | 3 | 16.7750 | 17.0573 | 42.6 |
| | 4 | 16.1457 | 16.5937 | 41.5 |
| | 5 | 16.1401 | 16.4017 | 41.0 |
| | 6 | 17.2194 | 17.5126 | 43.8 |
| 16 | 1 | 25.7099 | 26.1332 | 65.3 |
| | 2 | 25.1420 | 25.6837 | 64.2 |
| | 3 | 25.8069 | 26.2256 | 65.6 |
| | 4 | 25.0581 | 25.6374 | 64.1 |
| | 5 | 25.1374 | 25.5302 | 63.8 |
| | 6 | 26.5525 | 26.9857 | 67.5 |
| 24 | 1 | 36.6989 | 37.3320 | 93.3 |
| | 2 | 37.1410 | 37.8888 | 94.7 |
| | 3 | 37.3229 | 37.9631 | 94.9 |
| | 4 | 36.5355 | 37.3202 | 93.3 |
| | 5 | 36.3402 | 36.9390 | 92.3 |
| | 6 | 38.1316 | 38.7825 | 97.0 |

### Example 3

### Naltrexone Dissolution Profile for Oxycodone 20%

Six sample capsules of oxycodone/naltrexone beads, manufactured as described in Example 1, were tested for in vitro dissolution by placing the capsules in 0.1N HCl for 1 hour and then for 72 hours in 0.05M pH 7.5 phosphate. The results are shown in the table below:

| | | Naltrexone | Total naltrexone | % Naltrexone |
|---|---|---|---|---|
| Hour | Vessel | released (mg) | released (mg) | released |
| 1 | 1 | 0.0000 | 0.0000 | 0.0 |
| | 2 | 0.0000 | 0.0000 | 0.0 |
| | 3 | 0.0000 | 0.0000 | 0.0 |
| | 4 | 0.0000 | 0.0000 | 0.0 |
| | 5 | 0.0000 | 0.0000 | 0.0 |
| | 6 | 0.0000 | 0.0000 | 0.0 |
| 73 | 1 | 0.0123 | 0.0123 | 0.1 |
| | 2 | 0.0264 | 0.0264 | 0.2 |
| | 3 | 0.0000 | 0.0000 | 0.0 |
| | 4 | 0.0000 | 0.0000 | 0.0 |
| | 5 | 0.0356 | 0.0356 | 0.2 |
| | 6 | 0.0000 | 0.0000 | 0.0 |

### Example 4

### Oxycodone Dissolution Profile for Oxycodone 20% in Ethanol

Six sample capsules of oxycodone/naltrexone beads, manufactured as described in Example 1, were tested for in vitro dissolution by placing the capsules. The results are shown in the table below:

| | | Oxycodone | Total oxycodone | % Oxycodone |
|---|---|---|---|---|
| Hour | Vessel | released (mg) | released (mg) | released |
| 1 | 1 | 0.5478 | 0.5478 | 1.4 |
| | 2 | 0.5402 | 0.5402 | 1.4 |
| | 3 | 0.5534 | 0.5534 | 1.4 |
| | 4 | 0.7856 | 0.7856 | 2.0 |
| | 5 | 0.5387 | 0.5387 | 1.3 |
| | 6 | 0.5999 | 0.5999 | 1.5 |
| 2 | 1 | 3.1063 | 3.6541 | 9.1 |
| | 2 | 3.0507 | 3.5059 | 9.0 |
| | 3 | 3.2179 | 3.7713 | 9.4 |
| | 4 | 2.8804 | 3.6660 | 9.2 |
| | 5 | 3.0552 | 3.5939 | 9.0 |
| | 6 | 2.9985 | 3.5984 | 9.0 |
| 3 | 1 | 8.3591 | 8.9131 | 22.3 |
| | 2 | 8.2436 | 8.7899 | 22.0 |
| | 3 | 9.4686 | 10.0285 | 25.1 |
| | 4 | 7.8880 | 8.6794 | 21.7 |
| | 5 | 8.1764 | 8.7212 | 21.8 |
| | 6 | 9.0873 | 9.6932 | 24.2 |
| 4 | 1 | 14.4716 | 15.0423 | 37.6 |
| | 2 | 14.6603 | 15.2232 | 38.1 |
| | 3 | 14.6914 | 15.2702 | 38.2 |
| | 4 | 14.4200 | 14.2272 | 35.6 |
| | 5 | 14.5023 | 15.0635 | 37.7 |
| | 6 | 13.8563 | 14.4804 | 36.2 |
| 6 | 1 | 23.7323 | 24.3321 | 60.8 |
| | 2 | 24.4744 | 25.0667 | 62.7 |
| | 3 | 25.0990 | 25.7073 | 64.3 |
| | 4 | 23.9003 | 24.7344 | 61.8 |
| | 5 | 27.5089 | 28.0992 | 70.2 |
| | 6 | 25.8368 | 26.4888 | 66.2 |
| 8 | 1 | 30.4478 | 31.0953 | 77.7 |
| | 2 | 30.2939 | 30.9354 | 77.3 |
| | 3 | 30.7770 | 31.4358 | 78.6 |
| | 4 | 30.4854 | 31.3676 | 78.4 |
| | 5 | 29.4187 | 30.0644 | 75.2 |
| | 6 | 31.8032 | 32.5071 | 81.3 |
| 10 | 1 | 35.8945 | 36.6034 | 91.5 |
| | 2 | 34.9773 | 35.6799 | 89.2 |
| | 3 | 41.4177 | 42.1386 | 105.3 |
| | 4 | 37.0529 | 37.9966 | 95.0 |
| | 5 | 35.4227 | 36.1277 | 90.3 |
| | 6 | 35.2818 | 36.0499 | 90.1 |
| 12 | 1 | 40.9569 | 41.7383 | 104.3 |
| | 2 | 43.2000 | 43.9733 | 109.9 |
| | 3 | 39.9228 | 40.7274 | 101.8 |
| | 4 | 42.3140 | 43.3325 | 108.3 |
| | 5 | 38.1232 | 38.8997 | 97.2 |
| | 6 | 39.5107 | 39.5107 | 98.8 |

### Example 5

### In Vivo Single Dose Study of 20% Oxycodone Formulation

This study was an open-label, single-dose, randomized, 2-period crossover study in healthy volunteers. Twenty-four (24) subjects were enrolled and randomly assigned to 1 of 2 treatment sequences. Each subject received both treatments over the course of the study. Twenty-two (22) subjects completed both dosing periods including all post dose pharmacokinetic (PK) assessments.
Treatment A=4×5 mg oxycodone HCl IR tablets (total oxycodone HCl dose=20 mg) (reference)
Treatment B=1×oxycodone HCl (40 mg) and naltrexone HCl (8 mg) ER capsule (ALO-02) (test)

Subjects completed a Screening Phase, a Treatment Phase consisting of two Dosing Periods, and an End-of-Study Phase. The Screening Phase was conducted on an outpatient basis within 30 days prior to the start of the Treatment Phase.

During each Dosing Period, subjects were admitted to the clinical research unit (CRU) on the evening before dosing (Day-1). Subjects were dosed on Day 1 of each Dosing Period and confined in the CRU for 48 hours (discharged on Day 3).

Serial sampling of venous blood was performed on an inpatient basis during the initial 48 hours after dosing and on an outpatient basis thereafter out to 120 hours post dose. Vital signs, adverse event (AE) assessments, clinical laboratory assessments, and pulse oximetry were performed at specified times. Subjects were discharged from the CRU on Day 3 after the 48-hour post dose sample was obtained and all clinical assessments were complete to the satisfaction of the Investigator. Subjects returned to the CRU for blood sampling on an outpatient basis out to 120 hours post dose. Subjects then checked into the clinic for Dosing Period 2, following a washout period of at least 7 days. At the end of Dosing Period 2 (End of Study), a final safety assessment was performed.

A total of 24 healthy adult male and female subjects (30%-60% female) were enrolled to insure a completed minimum of 18 subjects. Twenty-four (24) subjects were enrolled, and 22 subjects completed both Dosing Periods. For pharmacokinetic (PK) analysis, the data from the 24 subjects who fully completed at least 1 Dosing Period were included in the PK population listings and summaries, the statistical analyses of the treatment comparisons, and figures for oxycodone, dose-normalized oxycodone, and noroxycodone. Subject #1 and Subject #21 received both treatments, but experienced vomiting <2 hours following dosing with the IR tablets (reference) during Periods 1 and 2, respectively. Subject #21 was discontinued from the Dosing Period 2, and Subject #1 was dosed in Period 2. These data were excluded from the summary statistics for the affected treatment. Subjects #2, #10, and #21 experienced vomiting in Dosing Period 1 after receiving the ER capsule (test) and these data were excluded from the summary statistics for the affected treatment. Subject #1 returned for Period 2 and was dosed per protocol, this subject vomited post dose following the ER capsule (test). Pharmacokinetic data from this subject (Period 2) were included in the summary statistics since the vomiting episode occurred within a minute of the end of the dosing interval of 12 hours. All 24 subjects were included in the safety analysis.

The PK parameters calculated for oxycodone and noroxycodone included maximum observed plasma concentration (Cₘₐₓ), areas under the plasma concentration-time curve (AUCₗₐₛₜ and AUC_{inf}), areas under the first moment curve (AUMCₗₐₛₜ and AUM_{inf}), time-to-maximum observed plasma concentration (Tₘₐₓ), half-life (T_{1/2}), the apparent terminal elimination rate constant (λ_{z}), and mean transit time (MTT). No PK parameters could be calculated for naltrexone since only two subjects demonstrated any measurable levels of naltrexone and only 4 subjects had the PK parameters estimated for 6-β-naltrexol (Cₘₐₓ, AUCₗₐₛₜ, and AUC_{inf} only).

Descriptive statistics were provided for oxycodone, noroxycodone, and 6-β-naltrexol concentrations and for the PK parameters. Analyses of variance (ANOVA) were performed on dose-normalized In-transformed plasma oxycodone PK parameters AUCₗₐₛₜ, AUC_{inf}, and Cₘₐₓ. PROC MIXED of SAS® (Version 9.1.3) was used with sequence, treatment, and period as fixed effects, and subject nested within sequence as a random effect. Geometric least-squares means (LSM), mean ratios, and 90% confidence intervals (CIs) were presented. The comparison of interest was the test ER capsule (ER1×40 mg oxycodone HCl and naltrexone HCl capsule) versus the reference IR tablets (IR 4×5 mg oxycodone HCl tablets, dose-normalized to 40 mg oxycodone HCl).

Safety assessments included the incidence, intensity, relationship to study drug, and seriousness of AEs, and changes in vital signs, 12-lead electrocardiograms (ECCs), clinical laboratory test values (chemistry, hematology, urinalysis), and physical examinations.

Adverse events were coded using the Medical Dictionary for Regulatory Activities (MedDRA®) Version 12.1. The incidences of treatment-emergent adverse events (TEAEs) were tabulated and compared across treatments. Descriptive summaries for clinical laboratory, vital signs, and ECG results were provided.

### Plasma Oxycodone

Descriptive statistics for PK parameters fbr oxycodone in plasma are presented in the following table:

Pharmacokinetic Parameters of Plasma Oxycodone

| | | 20 mg IR tablets | |
|---|---|---|---|
| | | (Reference) | |
| | 20 mg IR | Dose-Normalized | 40 mg ER |
| | tablets | to 40 mg | capsule |
| | (Reference) | Treatment A | (Test) |
| Pharma- | Treatment A | Arithmetic | Treatment B |
| cokinetic | Arithmetic | Mean ± | Arithmetic Mean ± |
| Parameters | Mean ± SD (N) | SD (N) | SD (N) |
| Cₘₐₓ | 38.9 ± 7.30 | 77.8 ± 14.6 | 22.6 ± 6.82 |
| (ng/mL) | (22) | (22) | (21) |
| Tₘₐₓ (h) | 1.00 | 1.00 | 14.0 |
| | (0.499, 3.98) | (0.499, 3.98) | (10.0, 24.0) |
| | (22) | (22) | (21) |
| AUCₗₐₛₜ | 245.9 ± 71.98 | 491.8 ± 144.0 | 570.8 ± 169.3 |
| (ng * h/mL) | (22) | (22) | (21) |
| AUC_{inf} | 252.7 ± 73.87 | 505.5 ± 147.7 | 627.0 ± 189.9 |
| (ng * h/mL) | (22) | (22) | (20) |
| AUMCₗₐₛₜ | 1434.6 ± 623.56 | 2869.2 ± 1247.1 | 12096 ± 3766.8 |
| (ng * h²/mL) | (22) | (22) | (21) |
| AUMC_{inf} | 1611.3 ± 702.63 | 3222.6 ± 1405.3 | 16085 ± 5248.7 |
| (ng * h²/mL) | (22) | (22) | (20) |
| t_{1/2} (h) | 3.74 ± 0.547 | 3.74 ± 0.547 | 12.0 ± 1.82 |
| | (22) | (22) | (20) |
| lambda z | 0.189 ± 0.0276 | 0.189 ± 0.0276 | 0.0588 ± 0.00857 |
| (1/h) | (22) | (22) | (20) |
| MTT (h) | 6.21 ± 0.967 | 6.21 ± 0.967 | 25.6 ± 2.40 |
| | (22) | (22) | (20) |

Treatment A = 4 × 5 mg oxycodone IR tablets (total oxycodone HCl dose = 20 mg) (reference)
Treatment B = 1 × oxycodone HCl (40 mg) and naltrexone HCl (8 mg) ER capsule ALO-02 (test)
Tₘₐₓ is presented as median (minimum, maximum)
Subjects #1 and #21 postdose Treatment A and Subjects #2, #10, and #21 postdose Treatment B were excluded from the summary statistics and statistical analysis due to vomiting.
C₂₄ was 14.3 ng/ml

The average values for oxycodone Cₘₐₓ, and Tₘₐₓ indicate that the rate of absorption of oxycodone from the ER capsule was substantially slower than that from the IR tablets, as evidenced by the lower mean Cₘₐₓ value (22.6 ng/mL versus 77.8 ng/mL) and prolonged median Tₘₐₓ (14.0 hours versus 1.0 hour) compared with the dose-normalized PK data for the tablets. With respect to mean values for AUCₗₐₛₜ and AUC_{inf}, there was no evidence of a fall-off in oxycodone bioavailability with the ER capsule. AUCs for the ER capsule were on average slightly higher than those for the IR tablets. Taking into consideration that different doses were utilized, the mean bioavailability results indicate that the overall delivery of oxycodone from the ER capsule was at least comparable to that of the commercial IR tablets.

The elimination phase appeared to be well characterized for each treatment, with mean half-life values of 12.0 hours and 3.7 hours for the ER capsule and IR tablets, respectively. Likewise, the mean oxycodone transit time (MTT) for the ER capsule was substantially more prolonged than that for IR tablets (25.6 hours versus 6.2 hours).

### Statistical Analysis for Oxycodone in Plasma (Dose-Normalized PK Parameters)

The ANOVA was performed to compare the PK parameters Cₘₐₓ, AUCₗₐₛₜ, and AUC_{inf} between Treatment B (40 mg ER capsule, test) and Treatment A (20 mg IR tablets, reference, values dose-normalized to 40 mg). The results of the statistical comparison are presented in the following table.

Statistical Analyses of Oxycodone PK Parameters (Dose-Normalized)

| | Geometric LSM | | | |
|---|---|---|---|---|
| | Treatment A | | | |
| | 20 mg | | | |
| | IR tablets | | | |
| Pharma- | (values dose- | Treatment B | Ratio | |
| cokinetic | normalized | 40 mg ER | B/A* | 90% CI* |
| Parameter | to 40 mg) | capsule | (%) | (%) |
| Cₘₐₓ | 77.320 | 21.525 | 27.84 | (25.06, 30.92) |
| (ng/mL) | | | | |
| AUCₗₐₛₜ | 484.917 | 531.264 | 109.56 | (104.66, 114.68) |
| (ng · h/mL) | | | | |
| AUC_{inf} | 498.180 | 593.048 | 119.04 | (112.82, 125.61) |
| (ng · h/mL) | | | | |

Treatment A = 4 × 5 mg oxycodone HCl IR tablets (total oxycodone HCl dose = 20 mg) (Reference)
Treatment B = 1 × oxycodone HCl (40 mg) and naltrexone HCl (8 mg) ER capsule (ALO-02) (test)
*= 90% CI and % Mean Ratios were dose-normalized to 40 mg oxycodone HCl and In-transformed prior to analysis.
Source: Table 14.2.1.8

Based on the geometric LSM ratio estimates, the results of the statistical analysis indicate that the Cₘₐₓ, for the ER capsule was only 27.8% of that for the IR tablets, indicating that the peak concentration of oxycodone had been blunted by the extended-release technology by approximately 72%. There was no evidence of a fall-off in bioavailability with the ER test capsule in comparison with the commercial IR reference tablets. In fact, the AUCₗₐₛₜ was 9.56% higher and the AUC_{inf} was 19.04% higher for the ER capsules compared with IR tablets. These slight differences in overall bioavailability (i.e., AUC) are not considered clinically significant in view of the number of subjects and different doses that were utilized in the present study.

### Plasma Noroxycodone

Descriptive statistics for PK parameters for noroxycodone in plasma are presented in the following table.

Pharmacokinetic Parameters of Plasma Noroxycodone

| | 20 mg IR tablets | 40 mg ER capsule |
|---|---|---|
| | (Reference) | (Test) |
| | Treatment A | Treatment B |
| Pharmacokinetic | Arithmetic Mean ± SD | Arithmetic Mean ± SD |
| Parameters | (N) | (N) |
| Cₘₐₓ (ng/mL) | 24.9 ± 6.26 | 13.4 ± 4.81 |
| | (22) | (21) |
| Tₘₐₓ (h) | 1.00 | 14.0 |
| | (0.499, 4.00) | (12.0, 24.0) |
| | (22) | (21) |
| AUCₗₐₛₜ (ng * h/mL) | 254.5 ± 86.05 | 409.8 ± 144.1 |
| | (22) | (21) |
| AUC_{inf} (ng * h/mL) | 265.3 ± 84.48 | 467.0 ± 174.8 |
| | (22) | (16) |
| AUMCₗₐₛₜ (ng * h²/mL) | 2405.2 ± 1301.8 | 9568.2 ± 3587.4 |
| | (22) | (21) |
| AUMC_{inf} (ng * h²/mL) | 2810.6 ± 1315.1 | 14268 ± 5672.3 |
| | (22) | (16) |
| t_{1/2} (h) | 6.51 ± 1.22 | 14.5 ± 2.45 |
| | (22) | (16) |
| lambda z (1/h) | 0.110 ± 0.0211 | 0.0491 ± 0.00832 |
| | (22) | (16) |
| MTT (h) | 10.2 ± 1.93 | 30.2 ± 3.43 |
| | (22) | (16) |

Treatment A = 4 × 5 mg oxycodone IR tablets (total oxycodone HCl dose = 20 mg) (reference)
Treatment B = 1 × oxycodone HCl (40 mg) and naltrexone HCl (8 mg) ER capsule ALO-02) (test)
Tₘₐₓ is presented as median (minimum, maximum)
Subjects 1 and 21 postdose Treatment A and Subjects 2, 10, and 21 postdose Treatment B were excluded from the summary statistics due to vomiting.
Source: Tables 14.2.2.3 and 14.2.2.4

Compared with IR tablets (reference), the ER capsule (test) showed lower noroxycodone Cmax (although given in a twice higher dose of oxycodone) and longer Tₘₐₓ (14 hours versus 1 hour). The overall extent of noroxycodone exposure (AUC_{inf}) for the 40 mg ER capsule (test) was approximately 1.8 times higher than the noroxycodone exposure following oxycodone dose of 20 mg IR tablets (reference).

The elimination phase appeared well characterized with half-life values of 14.5 and 6.51 hours for the ER capsule (test) and IR tablets (reference), respectively. Noroxycodone MTT for the ER capsule (test) was 30 hours, compared to 10 hours for the IR tablets (reference). The median time to peak was 14 hours post dose for the ER capsule (test) versus 1 hour post dose for the IR tablets (reference).

### Plasma Naltrexone and 6-β-Naltrexol

Blood samples for plasma naltrexone and 6-β-naltrexol were collected up to 120 hours post-dose after administration of the ER capsule (8 mg naltrexone). Of the 288 naltrexone plasma samples collected, only 2 subjects had quantifiable plasma naltrexone concentrations above the assay's lower limit of quantitation (LLOQ) of 4 pg/mL. Subject #2 had a naltrexone concentration of 4.59 pg/mL at 120 hours post dose and Subject #17 had a naltrexone concentration of 5.13 pg/mL at 72 hours post dose. Overall, including the subjects who were dropped from the statistical analysis because of vomiting, 286 of the 288 naltrexone samples (99.3%) were reported as below the assay limit of quantitation.

In contrast to naltrexone, plasma concentrations of 6-β-naltrexol were quantified in 15 subjects. In general, the appearance of metabolite occurred at low levels within 48-120 hours of dosing and there were no detectable levels in any subject within the first 24 hours of dosing. For 6-β-naltrexol, 4 out of 24 subjects (Subjects 1, 4, 17, and 23) had more than 2 measurable concentrations and PK parameters were calculated for those subjects only.

Descriptive statistics for PK parameters for 6-β-naltrexol in plasma are presented in the following table.

Descriptive Statistics for 6-β-Naltrexol

| | Cₘₐₓ | Tₘₐₓ | AUCₗₐₛₜ |
|---|---|---|---|
| Statistic | (pg/mL) | (h) | (pg * h/mL) |
| N | 4 | 4 | 4 |
| Mean | 58.9 | 78.0 | 2347 |
| SD | 68.3 | 12.0 | 2286 |
| Median | 27.2 | 72.0 | 1373 |
| Minimum | 20.1 | 72.0 | 903.1 |
| Maximum | 161 | 96.0 | 5739 |

Source: Table 14.2.4.2

The highest observed 6-β-naltrexol plasma concentration was 161 pg/mL, occurring at 72 hours post dose in Subject #17 (Table 14.2.4.1). However, the average value for 6-β-naltrexol concentration was 12.52 pg/mL at 72 hours post dose and the median concentration was 0 pg/mL across all time points with the exception of 96 hours post dose (2.16 pg/mL). In general, the low levels of 6-β-naltrexol, in combination with only trace concentrations of naltrexone, suggest that naltrexone remains largely intact within the core throughout the product's gastrointestinal transit, which was a desirable outcome with respect to product performance.

There were no serious AEs (SAEs) reported during this study. One (1) subject was discontinued due to the AE of vomiting, considered drug-related. A total of 210 AEs were reported by 24 (100%) subjects, with slightly higher incidence following IR tablets (reference) versus ER capsule (test). Headache was the most common AE, reported by 15 (63%) subjects overall, followed by dizziness (54%), nausea (50%), and fatigue (50%). All AEs resolved without sequelac. Of the 210 AEs, 205 were mild in intensity and 5 were moderate. The Investigator considered 187 AEs to be related to the study drug. No clinically relevant or treatment-related differences in clinical laboratory, vital sign, or ECG parameters were observed.

### Conclusions

The overall extent of exposure of oxycodone for the 1×40 mg oxycodone HCl and naltrexone HCl ER capsule (test) was approximately 19% higher than the reference IR formulation (4×5 mg oxycodone HCl tablets dose-normalized to 40 mg). The Cₘₐₓ was approximately 72% lower for the ER capsule (test) compared to the IR tablets (reference).

The median times to peak oxycodone and noroxycodone concentrations were 14 hours post dose for the ER capsule (test) versus 1 hour post dose for the IR tablets (reference).

Half-life values (12.0 hours for oxycodone and 14.5 hours for noroxycodone) for the ER capsule (test) were found to be higher than the reference IR tablets (3.74 hours for oxycodone and 6.51 hours for noroxycodone).

After administration of oxycodone HCl ER capsule (test) containing naltrexone HCl in its inner core, the plasma concentrations of naltrexone were below the limit of quantitation with the exception of 2 subjects who each had one measurable value just above the quantifiable limit (4.00 pg/mL). Most 6-β-naltrexol plasma concentrations were below the limit of quantitation, and low levels of 6-β-naltrexol were observed in 15 subjects, between 48 and 120 hours post dose.

Overall, the PK results of this study indicate that ALO-02 can deliver therapeutic amounts of oxycodone compared with a commercial IR formulation of oxycodone and that naltrexone systemic exposure levels were low.

Single doses of both the oxycodone HCl IR tablets (reference) and oxycodone HCl and naltrexone HCl ER capsule (test) administered in this study appeared to be generally safe and equally well tolerated by these healthy male and female subjects. The most frequent AEs were those commonly associated with opioid administration, including headache, dizziness, nausea, and fatigue. The distribution of these AEs was similar or sometimes greater with the IR formulation despite the higher dose of the oxycodone with ER, suggesting that some AEs, such as euphoria, may have been associated with the peak concentration (Cₘₐₓ) of oxycodone rather than its overall exposure level (AUC). No clinically relevant or treatment-related differences in clinical laboratory, vital sign, or ECG parameters were observed.

### Example 6

### 12% Oxycodone Formulation

### Sieved Sugar Spheres

Prior to seal coating, the sugar spheres are sieved to remove undersize spheres. The acceptably sized sugar spheres are collected and used in the seal coating process.

### Seal-Coated Sugar Spheres

### Charge

600 to 710 μm mesh sugar spheres (˜30 mesh): 1700 g

| Seal Coating dispersion | Solution | Solids | Apply |
|---|---|---|---|
| SD3A Ethanol | 80.00% | — | 4533.3 g |
| Dibutyl sebacate NF | 0.50% | 2.50% | 28.3 g |
| Ethylcellulose 50 NF | 5.00% | 25.00% | 283.3 g |
| Magnesium stearate | 2.00% | 10.00% | 113.3 g |
| Talc USP | 12.50% | 62.50% | 708.3 g |
| Total | 100.00% | 100.00% | 5666.7 g |

The manufacturing of seal-coated sugar spheres involves preparation of the seal coating dispersion and spray coating of the dispersion onto the sieved sugar spheres.

The seal coating dispersion is prepared by first dissolving dibutyl sebacate and ethylcellulose in alcohol. Talc and magnesium stearate are then added and dispersed uniformly into the solution prior to the seal coating operation. Mixing is continued until all the dispersion is applied.

The seal coating dispersion is sprayed onto the sieved sugar spheres utilizing a Wurster insert in the fluid bed. The coat application is performed under pre-defined process parameter settings. After all the seal coating dispersion has been sprayed, alcohol is sprayed on the product to flush the pump lines and spray nozzles. Once the flushing is complete, the product spheres are dried, discharged, weighed and sieved. Oversized and undersized spheres are subsequently discarded. The acceptably sized spheres are further processed into the next step.

### Naltrexone Hydrochloride Pellets Overview

Manufacturing of naltrexone hydrochloride pellets starts with naltrexone hydrochloride (NT) drug layering onto the seal-coated sugar spheres to form naltrexone cores. Subsequently, these naltrexone cores undergo a two-step coating of the sequestering membrane (also called barrier coat). All drug layering and coat applications are performed in a fluid bed equipped with a Wurster insert. Curing is performed in an oven after each step of the barrier coating and sieving is conducted on the final cured finished pellets.

### NT Cores

### Charge

Seal Coated Sugar Spheres (−18/+30 mesh: 1700 g

| Naltrexone | | | |
|---|---|---|---|
| HCl Dispersion | Solutions | Solid | Apply |
| SD3A Ethanol | 63.07% | — | 534.5 g |
| Purified Water USP | 16.21% | — | 137.4 g |
| Ascorbic Acid USP | 1.16% | 5.61% | 9.8 g |
| HPC NF (75-150 cps) | 2.24% | 10.81% | 19.0 g |
| Naltrexone HCl USP | 11.81% | 57.01% | 100.1 g |
| Talc USP | 5.50% | 26.57% | 46.7 g |
| Total | 100.00% | 100.00% | 847.5 g |

### Naltrexone Cores

The naltrexone dispersion is first prepared by dissolving ascorbic acid and hydroxypropyl cellulose into alcohol and purified water. Naltrexone hydrochloride and talc are then added and dispersed uniformly into the solution. Mixing is continued until all the dispersion is applied.

The naltrexone dispersion is sprayed onto the seal-coated sugar spheres utilizing a Wurster insert in the fluid bed. The drug coat application is performed under pre-defined process parameter settings. After all the naltrexone dispersion has been sprayed, alcohol is sprayed on the product to flush the pump lines and spray nozzles. Once the flushing is complete, the product cores are dried and discharged.

### NT Intermediate Pellets

### Naltrexone HCl Cores (−18/+30 Mesh): 1700 g

| Intermediate dispersion | Solution | solids | Apply |
|---|---|---|---|
| SD3A Ethanol | 62.34% | — | 3249.8 g |
| Purified Water USP | 17.67% | — | 921 g |
| SLS NF | 0.64% | 3.20% | 33.4 g |
| Dibutyl sebacate NF | 0.96% | 4.79% | 49.9 g |
| Eudragit RS | 7.60% | 37.99% | 395.9 g |
| Talc USP | 10.80% | 54.02% | 563.0 g |
| Talc USP (dusting) | — | — | — |
| Total | 100.00% | 100.00% | 5212.9 g |

### NT Finished Pellets

### Charge

### Naltrexone HCl Intermediate Pellets (−16/+25 Mesh): 2000.0 g

| Intermediate dispersion | Solution | Solids | Apply |
|---|---|---|---|
| SD3A Ethanol | 62.34% | — | 3823.3 g |
| Purified Water USP | 17.67% | — | 1083.5 g |
| SLS NF | 0.64% | 3.20% | 39.2 g |
| Dibutyl sebacate NF | 0.96% | 4.79% | 58.7 g |
| Eudragit RS | 7.60% | 37.99% | 465.8 g |
| Talc USP | 10.80% | 54.02% | 662.3 g |
| Talc USP (dusting) | — | — | — |
| Total | 100.00% | 100.00% | 6132.8 g |

### Naltrexone Pellets (Intermedialte and Finished)

The barrier coating process is performed in two steps—the first step produces naltrexone intermediate pellets and the second step produces finished pellets.

The barrier coating dispersions for both the intermediate pellets and finished pellets are prepared in the same way. Sodium lauryl sulfate, dibutyl sebacate, ammonio methacrylate copolymer type B (Eudragit® RS) are first dissolved into alcohol and purified water. Talc is dispersed into the solution before barrier coating commences. Mixing is continued until all the dispersion is applied.

For the naltrexone intermediate pellets, the barrier coating dispersion is sprayed onto naltrexone cores utilizing a Wurster insert in the fluid bed. The coat application is performed under pre-defined process parameter settings. After all the barrier coating dispersion has been sprayed, alcohol is sprayed onto the product to flush the pump lines and spray nozzles. Once the flushing is complete, the product pellets are dried and dusted with talc. The intermediate pellets are then transferred onto oven trays for curing. After curing, the intermediate pellets are weighed and sieved. The oversized and undersized pellets are subsequently discarded. The acceptably sized naltrexone intermediate pellets are further processed to the finished naltrexone pellets.

For the finished naltrexone pellets, the barrier coating dispersion is sprayed onto the cured naltrexone intermediate pellets utilizing a Wurster insert in the fluid bed. The same procedures (spraying, alcohol flushing, drying, dusting, curing and sieving) as the intermediate pellets are followed. The oversized and undersized pellets are subsequently discarded. The acceptably sized finished naltrexone pellets are further processed to the next step.

### ALO-02 Cores

### Charge

### Naltrexone HCl Pellets (−14/+25 Mesh): 2250 g

| Oxycodone HCl | | | |
|---|---|---|---|
| Dispersion | Solutions | Solids | Apply |
| SD3A Ethanol | 80.05% | — | 2680.6 g |
| HPC NF (75-150 cps) | 2.73% | 13.69% | 91.5 g |
| Oxycodone HCL USP | 11.48% | 57.54% | 384.4 g |
| Talc USP | 5.74% | 28.77% | 192.2 g |

Oxycodone Hydrochloride Cores with Sequestered Naltrexone Hydrochloride

The manufacturing of oxycodone hydrochloride cores with sequestered naltrexone hydrochloride involves preparation of the oxycodone hydrochloride drug dispersion and spray coating of the dispersion onto naltrexone hydrochloride pellets.

The oxycodone hydrochloride drug dispersion is prepared by first dissolving hydroxypropyl cellulose into alcohol. Oxycodone hydrochloride is added and uniformly dispersed into the solution prior to drug layering. Mixing is continued until all the dispersion is applied.

The oxycodone hydrochloride drug dispersion is sprayed onto naltrexone hydrochloride pellets utilizing a Wurster insert in the fluid bed. The drug layer application is performed under pre-defined process parameter settings. After all the drug dispersion has been sprayed, alcohol is sprayed on the product to flush the pump lines and spray nozzles. Once the flushing is complete, the product pellets are dried and discharged. The cores are then weighed and sieved. The oversized and undersized cores are rejected. The final acceptably sized cores are further processed to the next step.

### ALO-02 Pellets

### Charge

### Oxycodone HCl Cores: 2000.0 g

| Top Coat | | | |
|---|---|---|---|
| Dispersion | Solution | Solids | Apply |
| SD3A Ethanol | 85.71% | — | 2454.5 g |
| Diethyl phthalate | 1.05% | 7.33% | 30.0 g |
| NF | | | |
| PEG 6000 | 1.84% | 12.86% | 59.6 g |
| Eudragit L100-55 | 0.74% | 5.20% | 21.3 g |
| Ethylcellulose 50 | 5.89% | 41.24% | 168.8 g |
| NF | | | |
| Talc USP | 4.77% | 33.38% | 136.6 g |
| Talc USP (dusting) | — | — | 11.6 g |
| Total | 100.00% | 100.00% | 2863.9 g |

Final Pellets—Oxycodone Hydrochloride Extended Release with Sequestered Naltrexone Hydrochloride

The manufacturing of oxycodone hydrochloride extended-release with sequestered naltrexone hydrochloride pellets involves preparation of the coating dispersion and spray coating of the dispersion onto oxycodone hydrochloride cores with sequestered naltrexone.

The coating dispersion is prepared by first dissolving diethyl phthalate, polyethylene glycol (PEG), methacrylic acid copolymer type C (Eudragit® L100-55), and ethylcellulose into alcohol. Tale is then added and uniformly dispersed into the coating solution. Mixing is continued until all the dispersion is completely sprayed.

Utilizing a Wurster insert in the fluid bed, the coating dispersion is sprayed onto oxycodone hydrochloride cores with sequestered naltrexone. The coat application is performed under pre-defined process parameter settings. After all the coating dispersion has been sprayed, alcohol is sprayed on the product to flush the pump lines and spray nozzles. Once the flushing is complete, the product pellets are dried and dusted with talc. The pellets are then weighed and sieved. The oversized and undersized pellets are rejected. The final acceptably sized pellets are further processed to the next step.

### Oxycodone Hydrochloride and Naltrexone Hydrochloride Extended Release Capsules

Target fill weight for individual capsule is calculated based on the fractional potency of oxycodone hydrochloride for the final pellets and the capsule strength. Acceptable weight limits are calculated and must be between ±5% of the target fill weight. Designated capsule shells and pellets are dispensed. The capsules are filled with pellets either manually or by an automated encapsulation machine.

The overall amount of each component by weight and percentage in a batch production as well as the weight of each component per capsule is presented in the table below:

Composition of Oxycodone Hydrochloride and
Naltrexone Hydrochloride Extended Release Capsules, 40 mg/4.8 mg

| Component | By weight (g) | % | mg/capsule | Function |
|---|---|---|---|---|
| Active Components: | | | | |
| Oxycodone hydrochloride | 263.4 | 10.99 | 40.00¹ | Active component |
| Naltrexone | 31.6 | 1.32 | 4.80¹ | Active component² |
| hydrochloride | | | | |
| Inactive Components: | | | | |
| Talc (1656) | 930.2 | 38.64 | 140.64 | Hydrophobic glidant |
| | | | | Anti-sticking agent |
| | | | | Glidant |
| | | | | Anti-static agent |
| Ammonio methacrylic | 360.7 | 15.05 | 54.77 | Sequestering |
| copolymer (Type B | | | | membrane polymer |
| Powder) | | | | component |
| (Eudragit ® RS PO) | | | | |
| Sugar spheres | 322.3 | 13.45 | 48.94 | Seed core |
| (25-30 mesh) | | | | |
| Ethylcellulose | 222.5 | 9.08 | 33.04 | Rate controlling |
| (50 cps) | | | | polymer component |
| | | | | Film coating agent |
| Hydroxypropyl | 68.7 | 2.87 | 10.43 | Film coating agent |
| cellulose | | | | |
| (75-150 cps) | | | | |
| (Klucel ® LF) | | | | |
| Polyethylene glycol | 52.6 | 2.13 | 7.76 | Rate controlling |
| (6000) | | | | polymer component |
| Dibutyl sebacate | 50.8 | 2.12 | 7.71 | Plasticizer |
| Sodium lauryl sulfate | 30.4 | 1.27 | 4.61 | Sequestering |
| | | | | membrane component |
| Diethyl phthalate | 30.0 | 1.21 | 4.42 | Plasticizer |
| Methacryiic acid | 21.3 | 0.86 | 3.14 | Rate controlling |
| copolymer | | | | polymer component |
| (Type C Powder) | | | | |
| (Eudragit ® L100-55) | | | | |
| Magnesium stearate | 21.5 | 0.90 | 3.26 | Hydrophobic agent |
| (vegetable sourced) | | | | |
| Ascorbic acid | 3.1 | 0.13 | 0.47 | pH controlling agent |
| Total Capsule Fill | 2409.4 | 100% | 364.00 | |
| Weight | | | | |

¹Amount charged to batch may be corrected for potency and/or moisture.
²Although naltrexone hydrochloride is an active component, the formulation is designed to sequester the naltrexone hydrochloride so that it is not released.

1. A composition comprises a plurality of multi-layer pellets comprising:
a. a water soluble core;
b. an antagonist containing layer comprising naltrexone HCl coating the core;
c. a sequestering polymer layer coating the antagonist containing layer;
d. an agonist layer comprising an opioid agonist coating the sequestering polymer layer, and
e. a controlled release layer coating the agonist layer;
wherein the naltrexone HCl comprises at least 10% wt to wt of the opioid agonist and wherein the agonist is substantially released and the naltrexone HCl is substantially sequestered upon administration to a human being.

2. The composition of claim 1 wherein the naltrexone HCl comprises from about 10% to about 30% wt to wt of the opioid agonist.

3. The composition of claim 1 wherein the naltrexone HCl comprises from about 10% to about 25% wt to wt of the opioid agonist.

4. The composition of claim 1 wherein the naltrexone HCl comprises from about 10% to about 20% wt to wt of the opioid agonist.

5. The composition of claim 1 wherein the opioid agonist is oxycodone.

6. A composition comprises a plurality of multi-layer pellets comprising:
a. a water soluble core;
b. an antagonist containing layer comprising naltrexone HCl coating the core;
c. a sequestering polymer layer coating the antagonist containing layer;
d. an agonist layer comprising an opioid agonist coating the sequestering polymer layer; and
e. a controlled release layer coating the agonist layer;
wherein the weight of the naltrexone HCl comprises at least 5% the combined weight of the water soluble core, antagonist layer and sequestering polymer layer and wherein the agonist is substantially released and the naltrexone HCl is substantially sequestered upon administration to a human being.

7. The composition of claim 6 wherein the weight of the naltrexone HCl comprises from about 5% to about 30% of the combined weight of the water soluble core, antagonist layer and sequestering polymer layer.

8. The composition of claim 6 wherein the weight of the naltrexone HCl comprises from about 6% to about 25% of the combined weight of the water soluble core, antagonist layer and sequestering polymer layer.

9. The composition of claim 6 wherein the weight of the naltrexone HCl comprises from about 7% to about 15% of the combined weight of the water soluble core, antagonist layer and sequestering polymer layer.

10. The composition of claim 6 wherein the weight of the naltrexone HCl comprises from about 8% to about 10% of the combined weight of the water soluble core, antagonist layer and sequestering polymer layer.

11. The composition of claim 6 wherein the opioid agonist is oxycodone.

12. A dosage form comprising oxycodone hydrochloride and sequestered naltrexone hydrochloride wherein the naltrexone hydrochloride is present in an amount that is about 10% to about 30% of amount of oxycodone hydrochloride on a weight basis wherein the dosage form sequesters 100% of the naltrexone hydrochloride as determined at 73 hours by first placing the composition in 500 ml of a 0.1N HCl solution for 1 hour at 37° C., using USP paddle method, 100 rotations per minute, and then placing the composition in 500 mL of a pH 7.5, 0.05M phosphate buffer for 72 hours at 37° C., using USP paddle method, 100 rotations per minute and then determining the amount of naltrexone hydrochloride sequestered.

13. The dosage form of claim 12 wherein the naltrexone hydrochloride is present in an amount that is about 12% the amount of oxycodone hydrochloride on a weight basis.

14. The dosage form of claim 12 wherein the naltrexone hydrochloride is present in an amount that is about 12% the amount of oxycodone hydrochloride on a weight basis.

15. A composition comprising a plurality of multi-layer pellets comprising
a. a water soluble core;
b. an antagonist layer comprising naltrexone HCl coating the core;
c. a sequestering polymer layer coating the antagonist containing layer;
d. an agonist layer comprising an opioid agonist coating the sequestering polymer layer; and
e. a controlled release layer coating the agonist layer
wherein the naltrexone HCl comprises at least 10% wt to wt of the opioid agonist and wherein the agonist is substantially released with a time to maximum observed plasma concentration (Tₘₐₓ) is more than about 10 hours and the naltrexone HCl is substantially sequestered upon administration to a human being.

16. The composition of claim 15 wherein the Tₘₐₓ is more than about 12 hours.

17. The composition of claim 15 wherein the Tₘₐₓ is more than about 14 hours.

18. The composition of claim 15 wherein the Tₘₐₓ is from about 10 hours to about 16 hours.

19. The composition of claim 15 wherein the Tₘₐₓ is from about 12 hours to about 16 hours.

20. A method of treating moderate to severe chronic pain in a patient in need thereof such comprising administering to the patient a multi-layer pharmaceutical composition comprising:
a. a water soluble core;
b. an antagonist layer comprising naltrexone HCl coating the core;
c. a sequestering polymer layer coating the antagonist containing layer;
d. an agonist layer comprising an opioid agonist coating the sequestering polymer layer; and
e. a controlled release layer coating the agonist layer
wherein the naltrexone HCl comprises at least 10% wt to wt of the opioid agonist and wherein the agonist is substantially released with a time to maximum observed plasma concentration (Tₘₐₓ) is more than about 10 hours and the naltrexone HCl is substantially sequestered upon administration to a human being.

21. A method of treating moderate to severe chronic pain in a patient in need thereof such comprising administering to the patient a multi-layer pharmaceutical composition comprising:
a. a water soluble core;
b. an antagonist layer comprising naltrexone HCl coating the core;
c. a sequestering polymer layer coating the antagonist containing layer;
d. an agonist layer comprising an opioid agonist coating the sequestering polymer layer; and
e. a controlled release layer coating the agonist layer
wherein the naltrexone HCl comprises at least 10% wt to wt of the opioid agonist and wherein the agonist is substantially released with a ratio of plasma concentration of the agonist at 24 hours after administration (C₂₄) to maximum observed plasma concentration (Cₘₐₓ) of between about 0.2 to about 0.8.
